(19) United States
(12) Patent Application Publication
(10) Pub. No.: US 2020/0277396 A1
(43) Pub. Date: Sep. 3, 2020

(54) Treatment Method

(71) Applicant: KIRA BIOTECH PTY LIMITED, Fortitude Valley, Queensland (AU)

(72) Inventors: Derek Nigel John Hart, Balmain (AU); Georgina Jane Clark, Balmain (AU); Edward Alan Abadir, Camperdown (AU); Xinsheng Ju, Dundas Valley (AU); Ziduo Li, Kellyville (AU)

(21) Appl. No.: 16/646,745

(22) PCT Filed: Sep. 12, 2018
(86) PCT No.: PCT/AU2018/050987

§ 371 Date: Mar. 12, 2020

(30) Foreign Application Priority Data
Sep. 13, 2017 (AU) 2017903726

(51) Int. Cl. C07K 16/28; G01N 33/50; G01N 33/574; A61P 35/00

(52) U.S. Cl. CPC C07K 16/2896; G01N 33/5011; G01N 33/57407; A61K 2039/505; C07K 2317/565; C07K 2317/55; C07K 2317/54; A61P 35/00

(57) ABSTRACT

The present invention relates to a method of treating lymphoma in a subject, and more particularly, for treating Hodgkin lymphoma (HL), and non-Hodgkin lymphoma (NHL). The method comprises administering to the subject an effective amount of a CD83 binding protein. The invention also relates to methods for diagnosing and assessing lymphoma in a subject.

The present application claims priority from Australian provisional application no. 2017903726, the entirety of which is incorporated herein by reference.

### FIELD

The present invention relates to a method for the treatment of lymphoma in a subject, and more particularly, for the treatment of Hodgkin lymphoma (HL), and non-Hodgkin lymphoma (NHL) such as Diffuse large B-cell lymphoma (DLBCL) and Mantle-Cell lymphoma (MCL).

### BACKGROUND

Lymphoma is a group of blood cell tumors that develop from lymphocytes. The two main categories of lymphomas are HL and NHL.

HL is a haematological malignancy caused by Hodgkin and Reed-Sternberg cells (HRS). NHL includes all lymphomas except HL. Currently, patients suffering from HL are treated with multi-agent chemotherapy and radiotherapy. While current therapies for HL have a significant rate of success, 25% of patients experience disease relapse when they become refactory to either primary or secondary chemotherapy, and survival remains substantially lower especially in elderly patients who cannot tolerate such therapy. Therapies for NHL have a lower rate of success than for HL. Almost 1 in every 2 people with NHL will have the DLBCL form of the lymphoma and a further 5-10% will have MCL.

New targeted therapies are still needed for treatment of lymphoma, especially in patients with poor risk characteristics.

### SUMMARY

A first aspect of the invention provides a method of treating lymphoma in a subject, comprising administering to the subject an effective amount of a CD83 binding protein.

An alternative first aspect of the invention provides use of a CD83 binding protein in the manufacture of a medicament for treating lymphoma in a subject; or a CD83 binding protein for use in treating or preventing lymphoma in a subject.

A second aspect of the invention provides a method of treating HL in a subject, comprising administering to the subject an effective amount of a CD83 binding protein.

An alternative second aspect of the invention provides use of a CD83 binding protein in the manufacture of a medicament for treating HL in a subject; or a CD83 binding protein for use in treating HL in a subject.

A third aspect of the invention provides a method of treating NHL in a subject, comprising administering to the subject an effective amount of a CD83 binding protein.

An alternative third aspect of the invention provides use of a CD83 binding protein in the manufacture of a medicament for treating NHL in a subject; or a CD83 binding protein for use in treating NHL in a subject.

A fourth aspect provides a method of treating mantle cell lymphoma (MCL) in a subject, comprising administering to the subject an effective amount of a CD83 binding protein.

An alternative fourth aspect provides use of a CD83 binding protein in the manufacture of a medicament for treating MCL in a subject; or a CD83 binding protein for use in treating MCL in a subject.

A fifth aspect provides a method of treating Diffuse large B-cell lymphoma (DLBCL) in a subject, comprising administering to the subject an effective amount of a CD83 binding protein.

An alternative fifth aspect provides use of a CD83 binding protein in the manufacture of a medicament for treating DLBCL in a subject; or a CD83 binding protein for use in treating DLBCL in a subject.

A sixth aspect provides a method of treating DLBCL, MCL or HL in a subject, comprising administering to the subject an effective amount of a CD83 antibody conjugate.

An alternative sixth aspect provides use of a CD83 antibody conjugate in the manufacture of a medicament for treating DLBCL, MCL or HL in a subject; or a CD83 antibody conjugate for use in treating DLBCL, MCL or HL in a subject.

A seventh aspect provides a method of treating DLBCL, MCL or HL in a subject, comprising administering to the subject an effective amount of a CD83 bing protein, wherein the CD83 binding protein is a Bi-specific T-cell engager.

An alternative seventh aspect provides use of a CD83 binding protein in the manufacture of a medicament for treating DLBCL, MCL or HL in a subject, wherein the CD83 binding protein is a Bi-specific T-cell engager (BiTE); or a CD83 binding protein for use in treating DLBCL, MCL or HL in a subject, wherein the CD83 binding protein is a Bi-specific T-cell engager (BiTE).

An eighth aspect provides a method of treating DLBCL, MCL or HL in a subject, comprising administering an affective amount of a CAR T cell, wherein the CAR T cell comprises a CD83 binding protein.

An alternative eighth aspect provides use of a CAR T cell comprising a CD83 binding protein in the manufacture of a medicament for treating DLBCL, MCL or HL in a subject; or a CAR-T cell comprising a CD83 binding protein for use in treating DLBCL, MCL or HL in a subject

A ninth aspect of the invention provides a method of diagnosing lymphoma in a subject, comprising determining whether CD83 is expressed by lymphocytes of the subject.

A tenth aspect provides a method of assessing the severity or stage of lymphoma in a subject, comprising determining the level of soluble CD83 (sCD83) in serum of the subject.

An eleventh aspect provides a kit for treating lymphoma in a subject, comprising a CD83 binding protein and instructions for use of the CD83 binding protein to treat lymphoma.

### BRIEF DESCRIPTION OF THE DRAWINGS

FIG. 1 shows that CD83 is expressed on HL cell lines.

FIG. 1(A) are histograms showing the results of analysis by flow cytometry of CD83 expression on KM-H2, L428 and HDLM-2 lymphoma cell lines, which were stained with HB15a-FITC, HB15e-FITC or 3C12C-FITC anti-CD83 mAbs, respectively. Grey histograms, isotype control; open histograms, anti-CD83 antibodies. CD30 staining was used as a positive control. These data are representative of three independent experiments with comparative results. FIG. 1(B) are histograms showing the results of analysis by flow cytometry of CD15, CD25, CD40 and CD274 (PD-L1) expression on KM-H2 cells.

FIG. 2 shows that CD83 is expressed on HRS cells in HL patients and a significant proportion of patients with DLBCL.

FIG. 2(A) is a microscope image (×200 magnification) showing staining of paraffin embedded lymph node biopsy samples of HL with anti-CD83 and anti-CD30 antibody (dark portions). One representative sample shown. FIG. 2(B) is a microscope image showing staining of paraffin embedded lymph node biopsy samples from diffuse large B-cell lymphoma (DLBCL) patients with anti-CD20, anti-CD83 and anti-CD3 antibody (dark regions) (at ×200 magnification). FIG. 2(C) is a pie chart showing the results of analysis of CD83 expression level in HRS cells of HL patients (n=35). High: CD83 positive in >90% HRS cells; moderate: 10-90% CD83⁺ in HRS cells; low: 10% CD83⁺ in HRS cells. FIG. 2(D) is an image of one representative sample of each expression group referred to FIG. 2(C) with high amplification (×200 magnification). Arrow indicates HRS cells expressing CD83.

FIG. 3 shows trogocytosis of CD83 molecule from HRS to T cells.

FIG. 3(A) is a graph showing the percentage of CD83 expression on CD3⁺ T cells following co-culture of T cells from healthy donor PBMCs with KM-H2 cells for 4 hours at a ratio of 1:5. CD83 expression on CD3⁺ T cells was analyzed by flow cytometry, data were from 5 experiments. FIG. 3(B) is a plot showing CD83 expression on CD3+ T cells following co-culture of T cells and KM-H2 cells, with or without transwells, for 4 hours. CD83 expression on T cells was analyzed by flow cytometry, one of three representative experiments shown. FIG. 3(C) is a plot showing CellVue Claret (Claret) expression on CD3⁺ T cells following labelling of KM-H2 cells with CellVue Claret and co-cultured with purified CD3⁺ T cells at ratio of 5:1 for 4 hours. CellVue Claret expression on T cells was analyzed by flow cytometry. Data representative of 3 experiments. FIG. 3(D) is a graph showing the level of PD-1 (CD279) expression on CD83⁺ trogocytosed T cells co-cultured with KM-H2 cells for 4 hours. Expression was determined by flow cytometry (n=4). p value of one-way ANOVA analysis shown. FIG. 3(E) are graphs showing the level of PD-1 expression on trogocytosed CD4⁺ T or CD8⁺ T cells after co-cultured with KM-H2 cells for 4 hours. Expression was analyzed by flow cytometry (n=4). p value of one-way ANOVA analysis shown. FIG. 3(F) are representative plots obtained from analysis of PD-1 expression on T cells using flow cytometry.

FIG. 4 shows that T cell proliferation is inhibited by soluble CD83 (sCD83) secreted by HL cells, and sCD83 activity is abolished by the addition of 3C12C.

FIG. 4(A) is a graph showing the concentration of sCD83 detected in supernatant (SN) from KM-H2, L428 cell lines and diagnostic sera from HL patients by ELISA. P-value of Mann-Whitney t-test was shown. FIG. 4(B) are graphs showing Proliferation Index (PI) for purified T cells which were labelled with CFSE and stimulated with CD2/CD3/CD28 microbeads (3:1) in the presence of 25% SN of KM-H2 or plus 3C12C (anti-CD83 mAb) (5 μg/ml) for 5 days. Cells were analyzed by flow cytometry and the Proliferation Index (PI) calculated for total CD3⁺, CD4⁺ and CD8⁺ T cells using Flow Jo (n=6). P-value of one-way ANOVA analysis is shown. FIG. 4(C) are histograms showing the results of flow cytometry analysis when different volumes (v/v) of KM-H2 supernatant were added to CD2/CD3CD28 microbead-stimulated CFSE labelled human T cells. T cells were collected and CFSE was analyzed by flow cytometry at day 5. The PI and Division Index (DI) were calculated as indicators for proliferation. Representative data from one of 3 similar experiments shown. FIG. 4(D) are histograms showing the results of flow cytometry analysis of CFSE labelled T cells stimulated with CD2/CD3/CD28 microbeads and then cultured in 25% (v/v) KM-H2 SN with or without antibody 3C12C (5 and 10 μg/ml). T cell proliferation was analyzed on day 5. FIG. 4(E) are histograms showing the results of flow cytometry analysis of CFSE labelled T cells stimulated with CD2/CD3/CD28 microbeads and then cultured with different concentrations of 3C12C only. 3C12C alone had no effect on proliferation of CFSE labelled T cells after CD2/CD3/CD28 microbead-stimulation.

FIG. 5 shows a time course of sCD83 in HL patients during chemotherapy.

FIG. 5 are graphs showing sCD83 levels in the sera of six HL patients during different cycles of chemotherapy examined by ELISA. Arrows indicate when PET-scans were performed and the results of complete response (CR), partial response (PR) or progressive disease (PD) are noted.

FIG. 6 shows 3C12C and 3C12C-monomethyl-auristatin E (MMAE) kills HL cell lines in vitro.

FIG. 6(A) is a graph showing the percentage of cytotoxicity when target cells KM-H2 or L428, labelled with Calcein-AM were co-cultured with effector cells (human PBMC) at E:T ratio of 25:1 with increasing 3C12C concentration from 0 μg/ml to 1 μg/ml at 37° C. for 3 hours. Supernatant was collected for fluorescence reading (excitation 485 nm, emission 538 nm) of released Calcein. ADCC activity was calculated (n=3). FIG. 6(B) is a graph showing the number of viable cells following culturing of KM-H2 or HL-60 cells with different concentrations of 3C12C-MMAE for 3 days. Viable cells by 7AAD staining with flow cytometry. The half maximal inhibitory concentration (IC₅₀) is shown.

FIG. 7 shows that 3C12C reduced B cells in non-human primates.

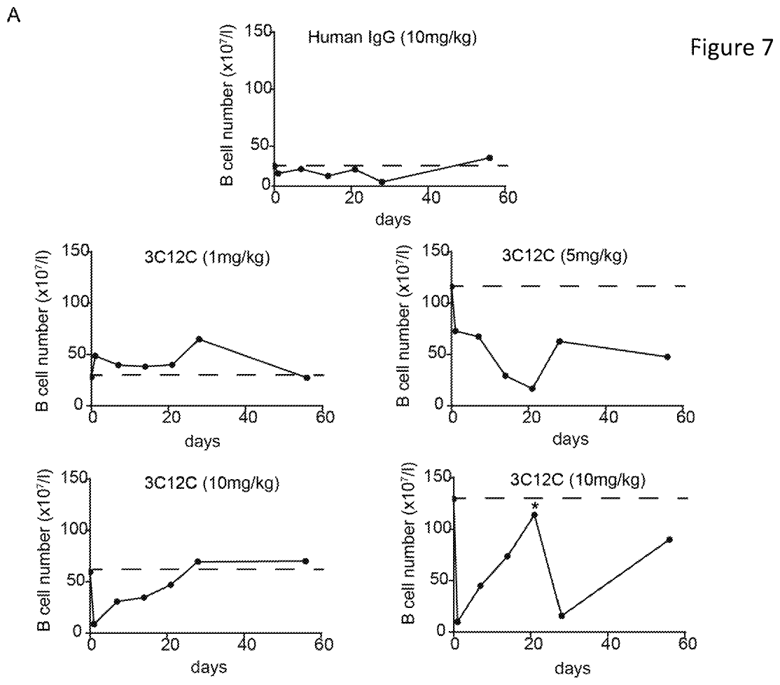

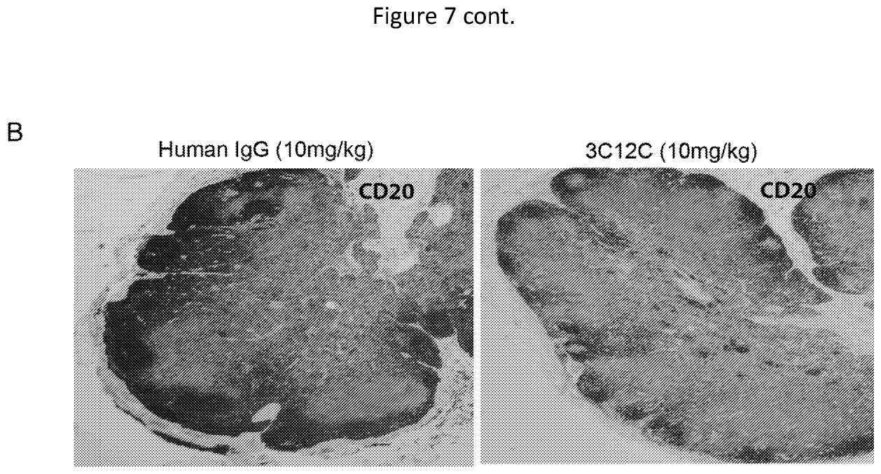

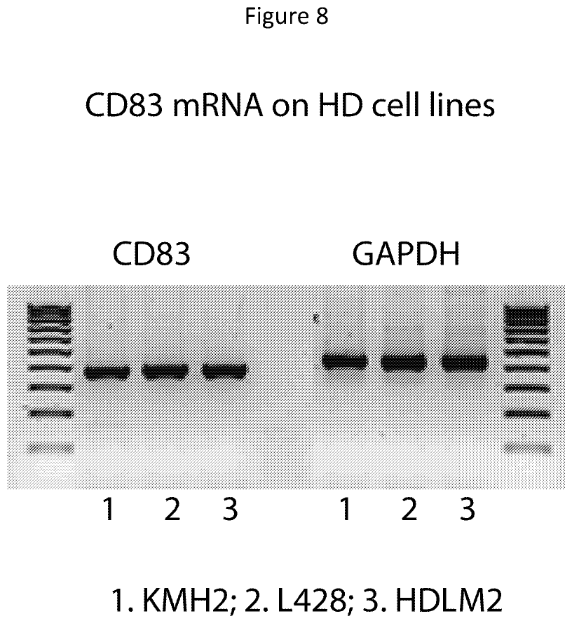

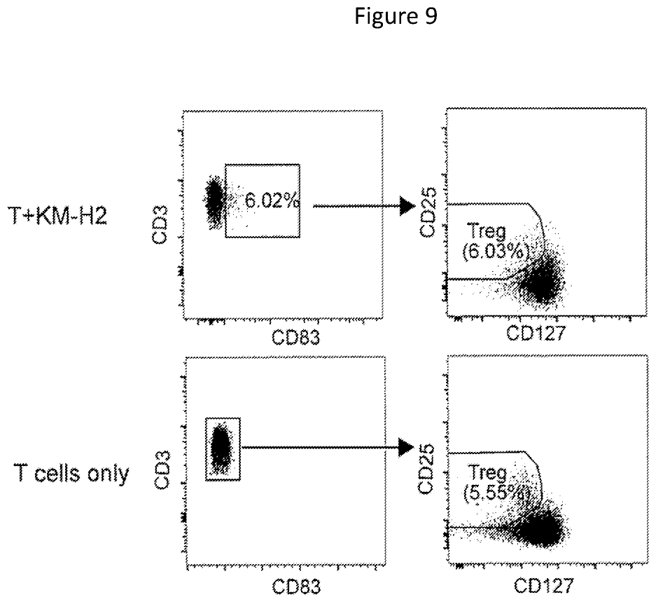

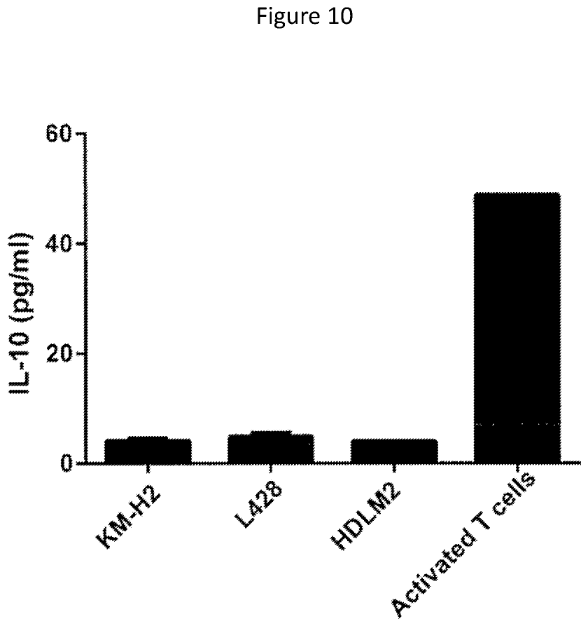

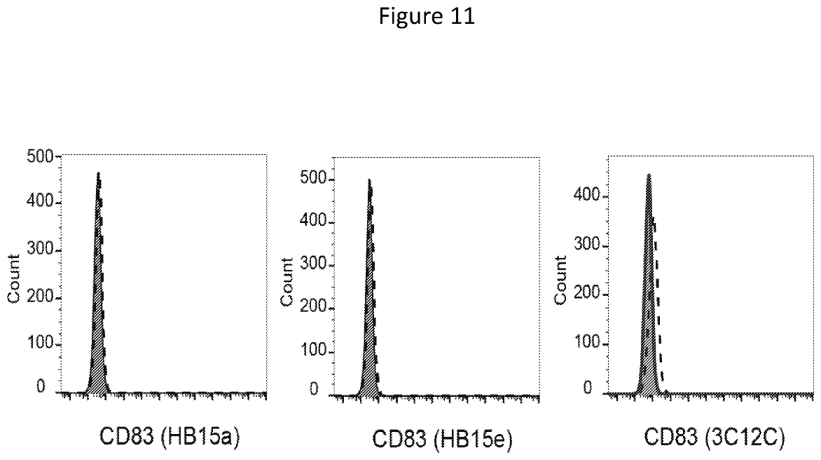

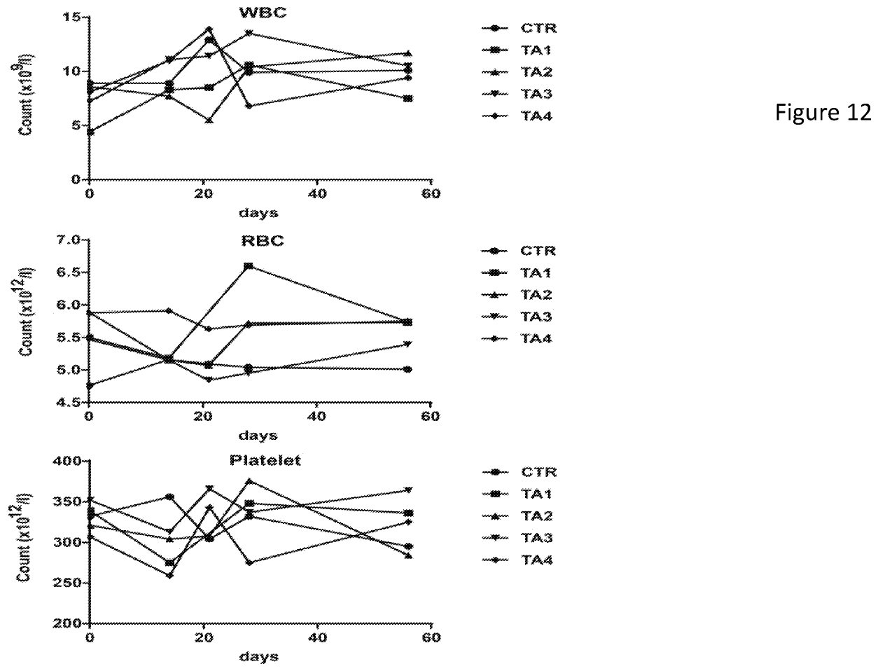

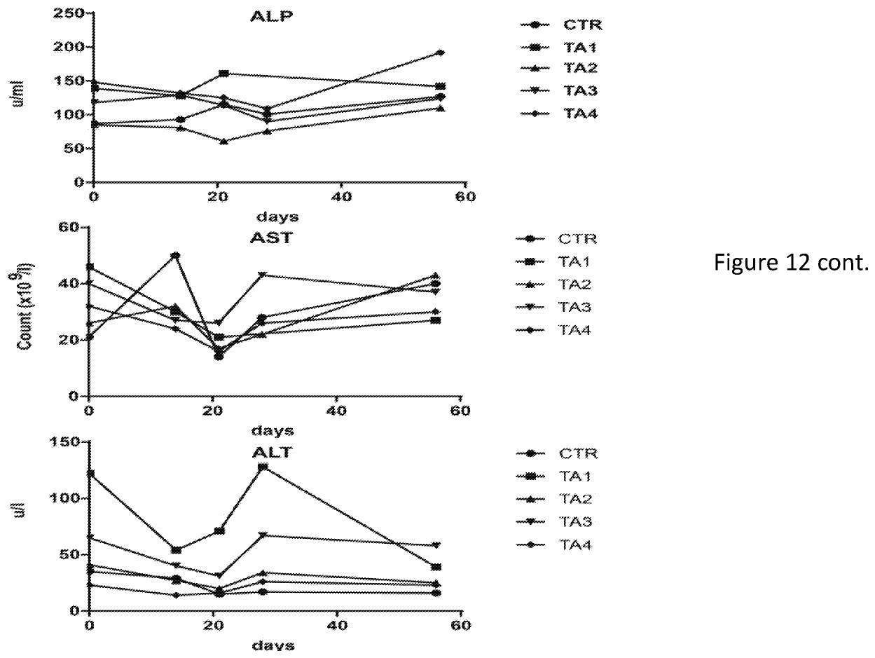

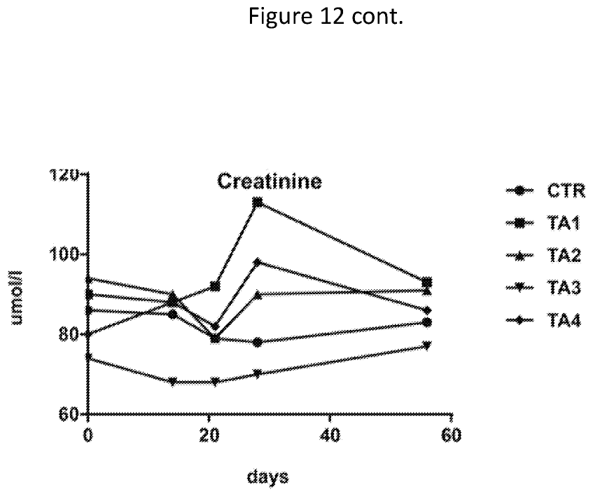

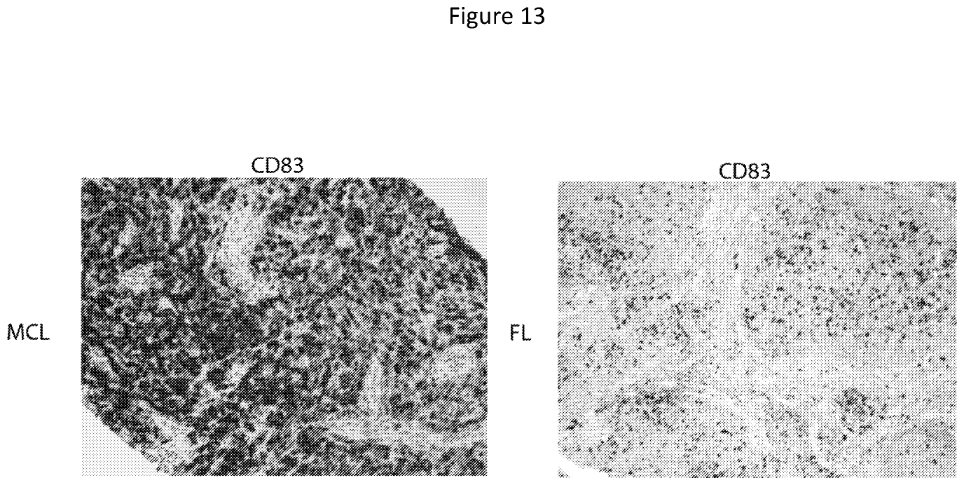

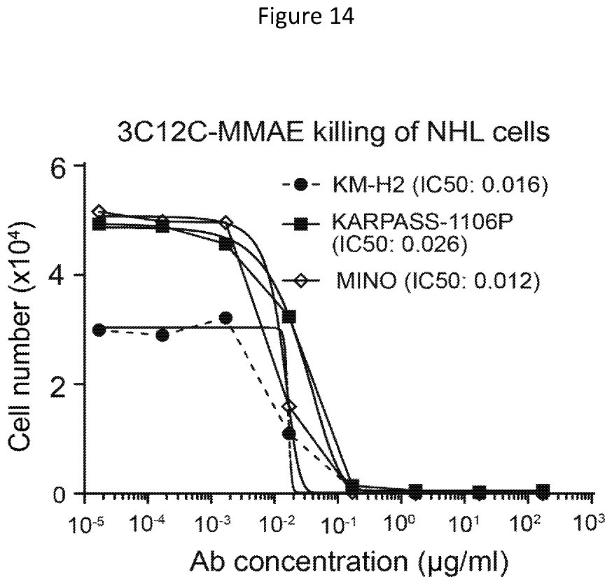

Five non-human primates were injected with 3C12C (1, 5, 10, 10 mg/kg, n=4) or human IgG (10 mg/kg, n=1) at days 0, 7, 14 and 21. A lymph node biopsy was taken at day 28 from 3C12C (10 mg/kg) and control treated animals. FIG. 7(A) is a graph showing number of CD19⁺ B cells from PBMC of 5 animals by flow cytometry. Dashed lines indicate the base cell number at day 0. * indicates one time point when WBC was extremely high in that animal. FIG. 7(B) is an image showing cells stained with anti-human CD20 mAb on paraffin embedded lymph node biopsy samples. Images from the animals receiving 10 mg/kg of 3C12C or human IgG are shown, the former showing a reduction in B cells.

FIG. 8 is an image showing the results of electrophoresis of HL cell line mRNA following amplification of CD83 and GAPDH mRNA by RT-PCR.

FIG. 9 shows that Treg cells from T cells co-cultured with KM-H2 cells.

Purified T cells were co-cultured with KM-H2 cells at ratio of 1:5 for 4 hours, the proportion of CD25⁺CD127^{low} Treg cells in CD83⁺ T cells were analysed by flow cytometry. T cells only culture condition was used as a control. Data from one of three experiments showing no increase in Treg cells.

FIG. 10 is a graph showing IL-10 levels in supernatant of HL lines.

Supernatants of KM-H2, L428 and HDLM2 were collected to measure the IL-10 level with CBA IL-10 beads assay. Low levels were demonstrated. Supernatant of PHA activated T cells was used as positive control.

FIG. 11 is graphs showing CD83 expression on HL60 line.

CD83 expression on HL60 was analysed with mouse anti-human CD83 mAb HB15a, HB15e or human anti-human CD83 mAb 3C12C by flow cytometry. Grey filled histograms were isotype controls for CD83 antibodies.

FIG. 12. are graphs showing 3C12C is safe in non-human primates.

Five non-human primates (Baboon) were injected with 3C12C (1 [TA1], 5 [TA2], 10 [TA3], 10 [TA4] mg/kg, n=4) or human IgG (10 [CTR] mg/kg, n=1) at day 0, 7, 14 and 21. Blood and serum samples were collected for blood cell counts (red cells (RBC), white cells (WBC) and platelets), liver (ALP, AST level) and kidney function (creatinine level) analysis. Data from the animals receiving 10 mg/kg of 3C12C or human IgG are shown. TA1=Animal receiving 1 mg/kg, TA2=Animal receiving 5 mg/kg, TA3=Animal receiving 10 mg/kg, TA4=Animal receiving 10 mg/kg, CTR=Control animal receiving 10 mg/kg human IgG.

FIG. 13 is a microscope image showing CD83 expression in MCL and FL from patients. Microscope image of CD83 expression (dark areas) on paraffin embedded lymph node biopsy samples from an MCL patient and a follicular lymphoma (FL) patient (×200 magnification).

FIG. 14 shows that 3C12C-MMAE kills DLBCL and MCL cell lines.

DLBCL line KARPASS-1106P or MCL line Mino cells were incubated with different concentrations of 3C12C-MMAE for 72 hours and then the viable cells were counted by flow cytometry. KM-K2 cells were used as a control. The half maximal inhibitory concentration (IC₅₀) is shown.

### DETAILED DESCRIPTION

The present disclosure relates to a method for treating lymphoma in a subject. Lymphoma is a group of blood cell tumors that develop from lymphocytes. The lymphoma may be HL or NHL.

In one embodiment, the lymphoma is HL. Hodgkin lymphoma is a lymphoma characterised by the presence of Hodgkin and Reed-Sternberg cells (HRS cells). HRS cells are identified typically as large bi-nucleated cells with prominent nucleoli and an CD45⁻, CD30⁺, CD15+^{+/-} immunophenotype. Typical characteristics of HRS cells include large size (20-50 micrometres), abundant, amphophilic, finely granular/homogeneous cytoplasm; two mirror-image nuclei (owl eyes) each with an eosinophilic nucleolus and a thick nuclear membrane (chromatin is distributed close to the nuclear membrane).

In another embodiment, the lymphoma is NHL. NHL is lymphoma not involving HRS cells.

In one embodiment, the NHL is MCL. Mantle cell lymphoma is a subtype of B-cell lymphoma, due to CD5 positive antigen-naive pre-germinal center B-cells within the mantle zone that surrounds normal germinal center follicles. Mantle cell lymphoma cells generally over-express cyclin D1.

In one embodiment, the NHL is diffuse large B-cell lymphoma (DLBCL).

In another embodiment, the NHL sub-type is Follicular lymphoma (FL) from a cell line, staining CD83 positive. Typically, the follicular lymphoma is from a cell line staining CD83 positive and comprising induced RNA proteins on the cell membrane.

The method of treating lymphoma comprises administering to the subject an effective amount of a CD83 binding protein.

CD83 is a single-pass type I membrane protein and member of the immunoglobulin superfamily. Three human transcript variants encoding different isoforms of CD83 have been identified. For the purposes of nomenclature and not limitation, the amino acid sequence of the human CD83 (hCD83) isoforms are shown in SEQ ID NO: 1 (NP_004224.1; isoform a), SEQ ID NO: 2 (NP_001035370.1; isoform b) and SEQ ID NO: 3 (NP_001238830.1; isoform c). Accordingly, in one example, the amino acid sequence of human CD83 comprises an amino acid sequence as shown in SEQ ID NO: 1, 2, or 3. Homologs of CD83 can be found in Pan troglodytes (XP 518248.2), Macaca mulatta (XP_001093591.1), Canis lupus familiaris (XP_852647.1), Bos Taurus (NP_001040055.1), Mus musculus (NP_033986.1), Rattus norvegicus (NP_001101880.1) and Gallus gallus (XP_418929.1).

CD83 is a marker of activated dendritic cells (DC), and is also expressed on activated B cell, T cells, macrophages, neutrophils etc. There are membrane-bound forms of CD83, and soluble forms of CD83 (sCD83).

### CD83 Binding Protein

A CD83 binding protein is a protein which is capable of specifically binding to CD83. The term “CD83 binding protein” includes a single polypeptide chain (i.e., a series of contiguous amino acids linked by peptide bonds), or a series of polypeptide chains covalently or non-covalently linked to one another (i.e., a polypeptide complex or protein), capable of specifically binding to CD83. For example, the series of polypeptide chains can be covalently linked using a suitable chemical or a disulphide bond. Examples of non-covalent bonds include hydrogen bonds, ionic bonds, Van der Waals forces, and hydrophobic interactions.

The CD83 binding protein typically comprises an antigen binding domain. An “antigen binding domain” is a region of an antibody that is capable of specifically binding to an antigen. The antigen binding domain of a CD83 binding protein specifically binds to CD83. An antigen binding domain typically comprises the complementarity determining region (CDR) 1, 2 and/or 3 of the heavy chain variable region, and/or the CDR 1, CDR2 and/or CDR3 of the light chain variable region, of an antibody. More typically, the antigen binding domain comprises CDR 1, 2 and 3 of the heavy chain variable region, and CDR 1, 2 and 3 of the light chain variable region, of an antibody. Still more typically, the antigen binding domain comprises a heavy chain variable region (V_{H}), and/or a light chain variable region (V_{L}), of an antibody. The antigen binding domain need not be in the context of an entire antibody, for example, it can be in isolation (e.g., a domain antibody) or in another form (e.g., scFv).

An “antibody” refers to a protein capable of specifically binding to one or a few closely related antigens (e.g., CD83) by an antigen binding domain contained within an Fv region of the antibody. An antibody comprises four chain antibodies (e.g., two light (L) chains and two heavy (H) chains), recombinant, or modified antibodies (e.g., chimeric antibodies, humanized antibodies, human antibodies, CDR-grafted antibodies, primatized antibodies, de-immunized antibodies, synhumanized antibodies, half-antibodies, and bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50 to 70 kDa each) covalently linked and two light chains (˜23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are ˜110 amino acids in length) and one or more constant domains at the C-terminus. The constant domain of the light chain (CL which is ˜110 amino acids in length) is aligned with and disulfide bonded to the first constant domain of the heavy chain (C_{H}1 which is 330 to 440 amino acids in length). The light chain variable region is aligned with the variable region of the heavy chain. The antibody heavy chain can comprise 2 or more additional C_{H} domains (such as, C_{H}2, C_{H}3 and the like) and can comprise a hinge region between the C_{H}1 and C_{H}2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In various embodiments, the antibody is humanized, synhumanized, chimeric, CDR-grafted or deimmunized.

As used herein, “variable region” refers to the portions of the light and/or heavy chains of an antibody as defined herein that is capable of specifically binding to an antigen and, includes amino acid sequences of complementarity determining regions (CDRs), that is, CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (i.e. CDR 1, CDR 2, and CDR 3) refers to the amino acid residues of an antibody variable region the presence of which are major contributors to specific antigen binding. Each variable region domain (V_{H} or V_{L}) typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk). According to the numbering system of Kabat, V_{H} FRs and CDRs are positioned as follows: residues 1 to 30 (FR1), 31 to 35 (CDR1), 36 to 49 (FR2), 50 to 65 (CDR2), 66 to 94 (FR3), 95 to 102 (CDR3) and 103 to 113 (FR4). According to the numbering system of Kabat, V_{L} FRs and CDRs are positioned as follows: residues 1 to 23 (FR1), 24 to 34 (CDR1), 35 to 49 (FR2), 50 to 56 (CDR2), 57 to 88 (FR3), 89 to 97 (CDR3) and 98 to 107 (FR4). The present disclosure is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system.

“Framework regions” (FRs) are those variable region residues other than the CDR residues.

As used herein, the term “Fv” refers to any protein, whether comprised of multiple polypeptides or a single polypeptide, in which a V_{L} and a V_{H} associate and form a complex having an antigen binding domain that is capable of specifically binding to an antigen. The V_{H} and the V_{L} which form the antigen binding domain can be in a single polypeptide chain or in different polypeptide chains. Furthermore, an Fv of the disclosure (as well as any protein of the disclosure) may have multiple antigen binding domains which may or may not bind the same antigen. This term shall be understood to encompass fragments directly derived from an antibody as well as proteins corresponding to such a fragment produced using recombinant means. Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, for example, C_{H}2 or C_{H}3 domain, for example, a minibody including other proteins like CAR T cell constructs.

An “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means.

An “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means.

An “F(ab′)2 fragment” of an antibody consists of a dimer of two Fab′ fragments held together by two disulfide bonds and is obtained by treating a whole antibody molecule with the enzyme pepsin, without subsequent reduction.

An “Fab₂” fragment is a recombinant fragment comprising two Fab fragments linked using, for example, a leucine zipper or a C_{H}3 domain.

A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a CD83 binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the antibody, will reduce the amount of labeled “A” bound to the antibody.

A protein that “specifically binds” or “binds specifically” to a particular antigen is a protein that reacts or associates more frequently, more rapidly, with greater duration and/or with greater affinity with the particular antigen than it does with alternative antigens. For example, a protein that specifically binds CD83 binds CD83 with greater affinity, avidity, more readily, and/or with greater duration than it binds to other antigens. In one example, “specific binding” of a CD83 binding protein to an antigen, means that the protein binds to the antigen with an equilibrium constant (K_{D}) of 100 nM or less, such as 50 nM or less, for example, 20 nM or less, such as, 15 nM or less or 10 nM or less or 5 nM or less or 1 nM or less or 500 pM or less or 400 pM or less or 300 pM or less or 200 pM or less or 100 pM or less.

As used herein, the term “epitope” (syn. “antigenic determinant”) means a region of an antigen to which a protein comprising an antigen binding domain of an antibody binds. This term is not necessarily limited to the specific residues or structure to which the protein makes contact. For example, this term includes the region spanning amino acids contacted by the protein and/or at least 5 to 10 or 2 to 5 or 1 to 3 amino acids outside of this region. In some examples, the epitope is a linear series of amino acids. An epitope may also comprise a series of discontinuous amino acids that are positioned close to one another when an antigen is folded, that is, a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three-dimensional structural characteristics, and/or specific charge characteristics. An epitope or peptide or polypeptide comprising same can be administered to an animal to generate antibodies against the epitope.

The method may employ any CD83 binding protein which is tolerated by the subject and which has a high affinity for CD83. CD83 binding proteins suitable for use in the method of the invention may be identified by screening libraries of antibodies or proteins comprising an antigen binding domain (e.g. comprising variable regions of antibodies) to identify CD83 binding proteins. Methods for screening libraries of proteins comprising antigen binding domains which specifically bind CD83 are described in, for example, WO2014/117220, and WO2016/061617.

In one embodiment, CD83 binding protein is an antibody.

In one embodiment, the antibody is a polyclonal antibody. Polyclonal antibodies may be prepared using methods that are known in the art. Polyclonal antibodies can be raised in a mammal, e.g., by one or more injections of an antigenic composition which is used to immunize the mammal. Typically, the antigenic composition is administered by multiple intravenous, subcutaneous or intraperitoneal injections. The immunization protocol may be readily selected by those skilled in the art. Methods for immunization and isolation of polyclonal antibodies are described in, for example, Antibodies: a Laboratory Manual by E. Harlow and D. Lane, 1988, Cold Spring Harbor Laboratory Press, Cold Spring Harbor, N.Y., chapter 5.

In one embodiment, the CD83 binding protein is a monoclonal antibody or antigen binding fragment thereof. Monoclonal antibodies may be prepared using methods know in the art, and described in, for example Antibodies: A Laboratory Manual by E. Harlow and D. Lane, 1988, Cold Spring Harbor Laboratory Press, Cold Spring Harbor, N.Y., chapters 5-7. A monoclonal antibody may be prepared, for example, by immunizing a mouse, hamster, or other appropriate host animal, with an antigen to elicit lymphocytes that produce or can produce antibodies that will specifically bind to the antigen. The antigen will typically be administered by administering an antigenic composition which includes, for example, a CD83 protein, such as that described in WO2016/061617. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

After the initial raising of antibodies to a CD83 protein, the antibodies can be sequenced and subsequently prepared by recombinant techniques to produce chimeric antibodies, such as humanized antibodies. Chimerisation of murine antibodies and antibody fragments are known to those skilled in the art. The use of antibody components derived from chimerized monoclonal antibodies reduces potential problems associated with the immunogenicity of murine sequence.

The variable domains from murine antibodies may be cloned using conventional techniques that are known in the art and described in, for example, Sambrook and Russell, Eds, Molecular Cloning: A Laboratory Manual, 3^{rd} Ed, vols. 1-3, Cold Spring Harbor Laboratory Press, 2001. In general, the variable light chain and variable heavy chain sequences for murine antibodies can be obtained by a variety of molecular cloning procedures, such as RT-PCR, 5′-RACE, and cDNA library screening. A chimeric antibody is an antibody protein that comprises the variable region, including the complementarity determining regions (CDRs) of an antibody derived from one species, typically a mouse antibody, while the constant domains of the antibody molecule are derived from another species, such as a human.

In some embodiments, the CD83 binding protein is a humanised antibody. A humanised antibody is a form of chimeric antibody in which the CDRs from an antibody from one species; e.g., a mouse antibody, are transferred from the heavy and light variable chains of the mouse antibody into human heavy and light variable domains (e.g., framework region sequences). The constant domains of the antibody molecule are derived from those of a human antibody.

The CD83 binding protein may thereof be a chimeric antibody. The chimeric antibody for use in the method described herein comprises the complementarity-determining regions (CDRs), and typically framework regions (FR), of a murine mAb which specifically binds a CD83 protein. The chimeric antibody may comprise the light and heavy chain constant regions of a human antibody. The use of antibody components derived from chimerized monoclonal antibodies reduces potential problems associated with the immunogenicity of murine constant regions. Humanization of murine antibodies and antibody fragments is known to those skilled in the art, and described in, for example, U.S. Pat. Nos. 5,225,539; 6,054,297; and U.S. Pat. No. 7,566,771. For example, humanized monoclonal antibodies may be produced by transferring murine complementary determining regions from heavy and light variable chains of the mouse immunoglobulin into a human variable domain, and then, substituting human residues in the framework regions of the murine counterparts. The use of human framework region sequences, in addition to human constant region sequences, further reduces the chance of inducing HAMA reactions. Antibodies can be isolated and purified from serum and hybridoma cultures by a variety of well-established techniques. Such isolation techniques include affinity chromatography with Protein-A Sepharose, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in Methods in Molecular Biology, vol. 10, pages 79-104 (The Humana Press, Inc. 1992).

In some embodiments, the CD83 binding protein is a fully humanised monoclonal antibody. Whereas, a humanised antibody is a form of chimeric antibody in which the CDRs from an antibody from one species; e.g., a mouse antibody, are transferred from the heavy and light variable chains of the mouse antibody into human heavy and light variable domains (e.g., framework region sequences). The constant domains of the antibody molecule are derived from those of a human antibody.

Antibodies which target CD83 can be characterized by a variety of techniques that are well-known to those of skill in the art. For example, the ability of an antibody to specifically bind to CD83 can be verified using, for example, an indirect enzyme immunoassay, flow cytometry analysis, ELISA or Western blot analysis.

A CD83 binding protein typically comprises the variable region of the heavy and/or light chain of an antibody, which specifically binds CD83. The portions of the variable heavy and/or light chain may be on separate polypeptide chains, such as Fv fragments, or in a single polypeptide chain in which light and heavy variable regions are connected by a peptide linker (“scFv proteins”). In one embodiment, the CD83 binding protein is an antigen binding fragment of an antibody. An antigen binding fragment of an antibody comprises the antigen binding domain of the antibody. Examples of antigen binding fragments include F(ab′)2, Fab′, Fab, Fv, sFv, scFv, and the like. Typically, the antigen binding fragment comprises the CDR1, 2 and/or 3 region of the variable heavy chain and/or the variable light chain. More typically, the antigen binding fragment comprises the CDR1, 2 and 3 region of the variable heavy chain and/or the variable light chain. Still more typically, the antigen binding fragment comprises the CDR1, 2 and 3 regions of the variable heavy chain and the CDR1, CDR2 and CDR3 of the variable light chain. Antigen binding fragments which recognize specific epitopes can be generated by known techniques. F(ab′)2 fragments, for example, can be produced by pepsin digestion of the antibody molecule. These and other methods are described, for example, by Coligan at pages 2.8.1-2.8.10 and 2.10-2.10.4. Alternatively, Fab′ expression libraries can be constructed to allow rapid and easy identification of Fab′ fragments with the desired specificity.

In some embodiments, the CD83 binding protein is a single chain Fv molecule (scFv). A single chain Fv molecule (scFv) comprises a VL domain and a VH domain. The VL and VH domains are typically covalently linked by a peptide linker (L) and fold to form an antigen binding site. While the V_{H} and V_{L} regions may be directly joined together, those skilled in the art will appreciate that the regions may be separated by a peptide linker consisting of one or more amino acids. Peptide linkers and their use are known in the art. Generally the peptide linker will have no specific biological activity other than to join the regions or to preserve some minimum distance or other spatial relationship between the V.sub.H and V.sub.L. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length.

Methods of making scFv antibodies are known in the art, and have been described in, for example, U.S. Pat. No. 5,260,203. For example, mRNA from B-cells from an immunized animal, or mRNA obtained from B lymphocytes purified from a panel of human donors, is isolated and cDNA is prepared. The cDNA is amplified using primers specific for the variable regions of heavy and light chains of immunoglobulins. The PCR products are purified, and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell. The scFv that specifically bind to the desired antigen are typically found by panning of a phage display library. Panning can be performed by any of several methods. Panning can conveniently be performed using cells expressing the desired antigen on their surface or using a solid surface coated with the desired antigen. Conveniently, the surface can be a magnetic bead. The unbound phage are washed off the solid surface and the bound phage are eluted.

Methods for preparing other antigen binding fragments are known in the art. For example, antigen binding fragments can also be prepared by proteolytic hydrolysis of a full-length antibody or by expression in E. coli or another host of the DNA coding for the fragment. An antibody fragment can be obtained by pepsin or papain digestion of full-length antibodies by conventional methods. For example, an antibody fragment can be produced by enzymatic cleavage of antibodies with pepsin to provide an approximate 100 Kd fragment denoted F(ab′)2. This fragment can be further cleaved using a thiol reducing agent, and optionally a blocking group for the sulfhydryl groups resulting from cleavage of disulfide linkages, to produce an approximate 50 Kd Fab′ monovalent fragment. Alternatively, an enzymatic cleavage using papain produces two monovalent Fab fragments and an Fc fragment directly.

Other methods of cleaving antibodies, such as separation of heavy chains to form monovalent light-heavy chain fragments, further cleavage of fragments, or other enzymatic, chemical or genetic techniques may also be used, so long as the fragments bind to the epitope that is recognized by the intact antibody.

In one embodiment, the CD83 binding protein is a bispecific antibody. Bispecific antibodies are monoclonal, preferably human or humanized, antibodies that have binding specificities for at least two different antigens or that have binding specificities for two epitopes on the same antigen.

In some embodiments, the bispecific antibodies are bi-specific T-cell engagers. Bi-specific T-cell engagers (BiTEs) are a class of artificial bispecific monoclonal antibodies. BiTEs are fusion proteins, typically comprising two single-chain variable fragments (scFvs) of different antibodies, or amino acid sequences from four different genes, on a single peptide chain. One of the scFvs binds to tumor antigen (e.g. CD83 target described herein) and the other generally to an effector cell, such as a T cell via the CD3 receptor. Method for preparing bispecific antibodies are described in, for example, Laszlo et al. Blood. 2014 Jan. 23; 123(4): 554-561; Loffler, Blood (2000), 95: 2098-103.

In another embodiment, the CD83 binding protein is a chimeric antigen receptor for chimeric antigen receptor T cells (CAR T cells). In this regard, nucleic acid encoding a polypeptide comprising an antigen binding domain, such as a scFv, in conjunction with a signaling molecule, can be used to transduce T cells to produce CAR T cells. The antigen binding domain expressed in the CAR T cells can recognize an antigen in a non-MHC restricted manner. Accordingly, expression of, for example, scFv encoding the antigen binding domain of anti-CD83 antibodies described herein, on the surface of T cells, may be effective in targeting CD83 on lymphoma cells. Methods for the preparation of CAR T cells are known in the art and described in, for example, Shannon et al. Blood, 25 Jun. 2015 Volume 125, No. 26: 4017-4023; O'Hear et al. (2015) Haematologica; 100(3): 336-344.

In one embodiment, the CD83 binding protein may be a human monoclonal antibody. Human monoclonal antibodies can be generated by immunizing transgenic mice carrying genes from the human immune system or can be derived from a phage human scFv library. For example, mice containing human immunoglobulin gene loci that encode unrearranged human heavy and light chain immunoglobulin sequences, may be immunized to produce human monoclonal antibodies. Examples of transgenic mice for production of human antibodies are known in the art and described in, for example, Lonberg et al. (1994) Nature 368: 856-859; Kellermann et al. (2002) Curr. Opin. Biotechnol. 13: 593-597; Tomizuka et al. (2000) PNAS 97: 722-727.

In one embodiment, the CD83 binding protein is a fully human antibody. Such an antibody may be produced from a human scFv and reformatted into an antibody with constant domains from a human antibody. For example, mRNA obtained from B lymphocytes purified from a panel of human donors may be used to produce human scFv as described herein. Human antibodies may be prepared by adding heavy and light chain constant regions to the heavy and light chain variable regions contained in the scFv sequences.

The antibodies described herein may be used to isolate other CD83 binding proteins, such as antibodies, which bind the same epitope, or overlapping epitope, by assessing cross-competition for the epitope. Cross-competition with the antibody or antigen binding fragments described herein can be assessed using methods known in the art, such as BIAcore analysis, flow cytometry, ELISA analysis.

Examples of CD83 binding proteins suitable for use in the method of the invention include anti-CD83 antibodies HB15a (available from Beckman and Coulter), HB15e (available from STEMCELL Technologies), monoclonal antibodies 3C12, 3C12B, 3C12C, 3C12D and 3C12E as described in WO2014/117220, and monoclonal antibodies 1F7, or derivatives thereof, as described in WO2016/061617.

In one embodiment, the CD83 binding protein comprises a heavy chain variable region (V_{H}) which comprises:
(i) a sequence which is at least 90% identical to the amino acid sequence shown in SEQ ID NO:10; or
(ii) three complementarity determining regions (CDRs) of the amino acid sequence shown in SEQ ID NO:10.

In one embodiment, the CD83 binding protein comprises:
(a) a heavy chain variable region (VH) which comprises:
(i) a sequence which is at least 90% identical to the amino acid sequence shown in SEQ ID NO:10; or
(ii) three complementarity determining regions (CDRs) of the amino acid sequence shown in SEQ ID NO:10; and
(b) a light chain variable region (V_{L}) which comprises:
   (i) a sequence which is at least 90% identical to any one of the amino acid sequences shown in SEQ ID NO: 12, 13, 11, 14, or 15; or
   (ii) three complementarity determining regions (CDRs) of any one of the amino acid sequences shown in SEQ ID NO: 12, 13, 11, 14, or 15; or
   (iii) a consensus sequence as shown in SEQ ID NO: 40 or
(iii) three CDRs, wherein the amino acid sequence of CDR1, CDR2, or CDR3 is a consensus sequence shown in SEQ ID NO: 37, 38, or 39.
In one embodiment, the CD83 binding protein comprises an antigen binding domain which comprises:
(a) a heavy chain variable region (VH) which comprises:
   (i) a sequence which is at least 90% identical to the amino acid sequence shown in SEQ ID NO:10; or
   (ii) three complementarity determining regions (CDRs) of the amino acid sequence shown in SEQ ID NO:10; and
(b) a light chain variable region which comprises:
   (i) a CDR1 sequence comprising the amino acid sequence of SEQ ID NO: 37, a CDR2 sequence comprising the amino acid sequence of SEQ ID NO: 38 and a CDR3 sequence comprising the amino acid sequence of SEQ ID NO: 39.

In one embodiment, the CD83 binding protein comprises an antigen binding domain which comprises:
(a) a heavy chain variable region which comprises a CDR1 sequence comprising the amino acid sequence of SEQ ID NO: 4, a CDR2 sequence comprising the amino acid sequence of SEQ ID NO: 5 and a CDR3 sequence comprising the amino acid sequence of SEQ ID NO: 6; and
(b) a light chain variable region which comprises a CDR1 sequence comprising the amino acid sequence of SEQ ID NO: 7, a CDR2 sequence comprising the amino acid sequence of SEQ ID NO: 8 and a CDR3 sequence comprising the amino acid sequence of SEQ ID NO: 9.

In one embodiment, the CD83 binding protein comprises an antigen binding domain which comprises a variable heavy chain comprising an amino acid sequence of SEQ ID NO: 10, and a variable light chain comprising an amino acid sequence of SEQ ID NO: 11.

In one embodiment, the CD83 binding protein is monoclonal antibody 3C12C as described in WO2014/117220.

In various other embodiments, the CD83 binding protein comprises an antigen binding domain which comprises:
(i) a V_{H} sequence as shown in SEQ ID NO:10 and a V_{L} sequence as shown in SEQ ID NO:12; or
(ii) a V_{H} sequence as shown in SEQ ID NO:10 and a V_{L} sequence as shown in SEQ ID NO:13; or
(iii) a V_{H} sequence as shown in SEQ ID NO:10 and a V_{L} sequence as shown in SEQ ID NO:14; or
(iv) a V_{H} sequence as shown in SEQ ID NO:10 and a V_{L} sequence as shown in SEQ ID NO:15; or
(v) a heavy chain sequence as shown in SEQ ID NO: 21 and a light chain sequence as shown in SEQ ID NO:16; or
(vi) a heavy chain sequence as shown in SEQ ID NO: 21 and a light chain sequence as shown in SEQ ID NO:17; or
(vi) a heavy chain sequence as shown in SEQ ID NO:21 and a light chain sequence as shown in SEQ ID NO:18; or
(vii) a heavy chain sequence as shown in SEQ ID NO:21 and a light chain sequence as shown in SEQ ID NO:19; or
(viii) a heavy chain sequence as shown in SEQ ID NO:21 and a light chain sequence as shown in SEQ ID NO:20; or
(ix) a V_{H} sequence as shown in SEQ ID NO:22 and a V_{L} sequence as shown in SEQ ID NO:23; or
(x) a V_{H} sequence as shown in SEQ ID NO:22 and a V_{L} sequence as shown in SEQ ID NO:24; or
(xi) a V_{H} sequence as shown in SEQ ID NO:22 and a V_{L} sequence as shown in SEQ ID NO:25;
(xii) a V_{H} sequence as shown in SEQ ID NO:22 and a V_{L} sequence as shown in SEQ ID NO:26;
(viii) a V_{H} sequence as shown in SEQ ID NO:22 and a V_{L} sequence as shown in SEQ ID NO:27;
(viii) a V_{H} sequence as shown in SEQ ID NO:22 and a V_{L} sequence as shown in SEQ ID NO: 28;
(viii) a V_{H} sequence as shown in SEQ ID NO:22 and a V_{L} sequence as shown in SEQ ID NO: 29;
(viii) a V_{H} sequence as shown in SEQ ID NO:22 and a V_{L} sequence as shown in SEQ ID NO: 30;
(viii) a V_{H} sequence as shown in SEQ ID NO:22 and a V_{L} sequence as shown in SEQ ID NO: 31;
(viii) a V_{H} sequence as shown in SEQ ID NO:22 and a V_{L} sequence as shown in SEQ ID NO: 32;
(viii) a V_{H} sequence as shown in SEQ ID NO:22 and a V_{L} sequence as shown in SEQ ID NO: 33; or
(viii) a V_{H} sequence as shown in SEQ ID NO:22 and a V_{L} sequence as shown in SEQ ID NO: 34; or
(vix) a heavy chain sequence as shown in SEQ ID NO: 35 and a light chain sequence as shown in SEQ ID NO: 36.

In one aspect, there is provided a method of treating lymphoma in a subject, comprising administering an effective amount of a CD83 binding protein which comprises a variable heavy chain comprising an amino acid sequence of SEQ ID NO: 10, and a variable light chain comprising an amino acid sequence of SEQ ID NO: 11.

Another aspect provides a method of treating HL in a subject, comprising administering an effective amount of a CD83 binding protein which comprises a variable heavy chain comprising an amino acid sequence of SEQ ID NO: 10, and a variable light chain comprising an amino acid sequence of SEQ ID NO: 11.

Another aspect provides a method of treating mantle cell lymphoma in a subject, comprising administering an effective amount of a CD83 binding protein which comprises a variable heavy chain comprising an amino acid sequence of SEQ ID NO: 10, and a variable light chain comprising an amino acid sequence of SEQ ID NO: 11.

Another aspect provides a method of treating DLBCL in a subject, comprising administering an effective amount of a CD83 binding protein which comprises a variable heavy chain comprising an amino acid sequence of SEQ ID NO: 10, and a variable light chain comprising an amino acid sequence of SEQ ID NO: 11.

Examples of nucleotide sequences encoding the light and heavy chains of antibodies described herein are shown in SEQ ID Nos: 41-59.

A summary of the sequence listing is set out below:

| SEQ | |
|---|---|
| ID NO | Description of sequence |
| 1 | amino acid sequence of Human CD83 isoform a |
| 2 | amino acid sequence of Human CD83 isoform b |
| 3 | amino acid sequence of Human CD83 isoform c |
| 4 | amino acid sequence of heavy chain CDR 1 of mAb 3C12.C |
| 5 | amino acid sequence of heavy chain CDR 2 of mAb 3C12.C |
| 6 | amino acid sequence of heavy chain CDR 3 of mAb 3C12.C |
| 7 | amino acid sequence of light chain CDR 1 of mAb 3C12.C |
| 8 | amino acid sequence of light chain CDR 2 of mAb 3C12.C |
| 9 | amino acid sequence of light chain CDR 3 of mAb 3C12.C |
| 10 | amino acid sequence of heavy chain variable region of |
| | mAb 3C12.C |
| 11 | amino acid sequence of light chain variable region of |
| | mAb 3C12.C |
| 12 | amino acid sequence of light chain variable region of |
| | mAb 3C12 |
| 13 | amino acid sequence of light chain variable region of |
| | mAb 3C12.B |
| 14 | amino acid sequence of light chain variable region of |
| | mAb 3C12.D |
| 15 | amino acid sequence of light chain variable region of |
| | mAb 3C12.E |
| 16 | amino acid sequence of light chain of mAb 3C12 |
| 17 | amino acid sequence of light chain of mAb 3C12.B |
| 18 | amino acid sequence of light chain of mAb 3C12.C |
| 19 | amino acid sequence of light chain of mAb 3C12.D |
| 20 | amino acid sequence of light chain of mAb 3C12.E |
| 21 | amino acid sequence of heavy chain of mAb 3C12 |
| 22 | amino acid sequence of heavy chain variable region of |
| | mAb 1F7 |
| 23 | amino acid sequence of light chain variable region of |
| | mAb 1F7 |
| 24 | amino acid sequence of light chain variable region of |
| | hFab4.1 |
| 25 | amino acid sequence of light chain variable region of |
| | hFab4.2 |
| 26 | amino acid sequence of light chain variable region of |
| | hFab4.3 |
| 27 | amino acid sequence of light chain variable region of |
| | hFab4.4 |
| 28 | amino acid sequence of light chain variable region of |
| | hFab4.5 |
| 29 | amino acid sequence of light chain variable region of |
| | hFab4.7 |
| 30 | amino acid sequence of light chain variable region of |
| | hFab4.8 |
| 31 | amino acid sequence of light chain variable region of |
| | hFab4.9 |
| 32 | amino acid sequence of light chain variable region of |
| | hFab4.10 |
| 33 | amino acid sequence of light chain variable region of |
| | hFab4.12 |
| 34 | amino acid sequence of light chain variable region of |
| | hFab4.18 |
| 35 | amino acid sequence of heavy chain of mAb 1F7 |
| 36 | amino acid sequence of light chain of mAb 1F7 |
| 37 | amino acid sequence of VL consensus sequence of CDR1 |
| | of 3C12 and derivatives |
| 38 | amino acid sequence of VL consensus sequence of CDR2 |
| | of 3C12 and derivatives |
| 39 | amino acid sequence of VL consensus sequence of CDR3 |
| | of 3C12 and derivatives |
| 40 | amino acid sequence of VL consensus sequence of 3C12 |
| | and derivatives |
| 41 | nucleotide sequence of 3C12 heavy chain |
| 42 | nucleotide sequence of 3C12 light chain |
| 43 | nucleotide sequence of 3C12.B light chain |
| 44 | nucleotide sequence of 3C12.C light chain |
| 45 | nucleotide sequence of 3C12.D light chain |
| 46 | nucleotide sequence of 3C12.F light chain |
| 47 | nucleotide sequence of 1F7 heavy chain variable region |
| 48 | nucleotide sequence of 1F7 light chain |
| 49 | nucleotide sequence of hFab4.1 light chain |
| 50 | nucleotide sequence of hFab4.2 light chain |
| 51 | nucleotide sequence of hFab4.3 light chain |
| 52 | nucleotide sequence of hFab4.4 light chain |
| 53 | nucleotide sequence of hFab4.5 light chain |
| 54 | nucleotide sequence of hFab4.7 light chain |
| 55 | nucleotide sequence of hFab4.8 light chain |
| 56 | nucleotide sequence of hFab4.9 light chain |
| 57 | nucleotide sequence of hFab4.10 light chain |
| 58 | nucleotide sequence of hFab4.12 light chain |
| 59 | nucleotide sequence of hFab4.18 light chain |

As further described in the Examples, the inventors have also analysed the killing effect of anti-human CD83 monoclonal antibody and their toxin conjugates and test their safety in non-human primate trial.

### Effector Function

In one embodiment, a CD83 binding protein may induce effector function.

As described herein, “effector function” refers to those biological activities (e.g., mediated by cells or proteins that bind to the Fc region) of an antibody that result in killing of a cell to which the antibody is bound. Examples of effector functions induced by antibodies include: complement dependent cytotoxicity (CDC); antibody-dependent-cell-mediated cytotoxicity (ADCC); antibody-dependent-cell-phagocytosis (ADCP); and B-cell activation.

“Antibody-dependent-cell-mediated cytotoxicity” or “ADCC” refers to lysis of antibody-bound target cells by effector cells (e.g., natural killer (“NK”) cells, neutrophils and/or macrophages) having Fc receptors that recognize the Fc region of the bound antibody. To assess ADCC activity of a molecule of interest, an in vitro ADCC assay may be performed. Useful effector cells for such assays include peripheral blood mononuclear cells (“PBMC”) and NK cells.

In one embodiment, the CD83 binding protein binds to CD83 on the surface of a cell in such a manner that it can induce an effector function, such as, ADCC and/or CDC.

In another embodiment, the CD83 binding protein has been engineered to improve induction of effector function by alteration of specific amino acids of the heavy chain of the antibody or by alteration of the carbohydrate moiety of the antibody Heavy chain.

Methods for determining effector function are known in the art and are described in, for example, Hellstrom et al. Proc. Natl Acad. Sci. USA 83: 7059-7063, 1986 and Bruggemann et al., J. Exp. Med. 166: 1351-1361, 1987; U.S. Pat. No. 7,317,091; Gazzano-Santoro et al., J. Immunol. Methods 202: 163, 1996). Other assays for assessing the level of ADCC induced by an immunoglobulin include ACTI™ nonradioactive cytotoxicity assay for flow cytometry (CellTechnology, Inc. CA, USA) or CytoTox 96® non-radioactive cytotoxicity assay (Promega, Wis., USA).

### Immunoconjugates

In one embodiment, the CD83 binding protein is an immunoconjugate. As used herein, an immunoconjugate is an antibody or antigen binding fragment thereof conjugated to a moiety, such as a therapeutic moiety and/or a diagnostic moiety.

In one embodiment, the CD83 binding protein is an immunoconjugate comprising a therapeutic moiety. A therapeutic moiety is a compound, molecule or atom which is useful in the treatment of a disease. Examples of therapeutic moieties include drugs, such as cytotoxic agents, such as chemotherapeutic agents; pro-apoptotic agents; radioisotopes; immunotoxins. A cytotoxic agent is a compound which is toxic to cells. Examples of cytoxotoxic agents include doxorubicin, cyclophosphamide, methotrexate, mustine, vincristine, procarbzine, prednisolone, bleomycin, vinblastine, dacarbazine, cyclophosphamide, Procarbazine, Paclitaxel, Irinotecan, Gemcitabine, Fluorouracil, Cytarabine, ozogamicin, adriamycin, etoposide, melphalan, mitomycin C, chloramuil, daunorubicin. Examples of radioisotopes include phosphorus-32, copper-67, arsenic-77, rhodium-105, palladium-109, silver-111, tin-1221, iodine-125, iodine-131, holmium-166, lutetium-177, rhenium-186, iridium-194, gold-199, astatium-211, yttrium-90, and bismuth-212. Examples of immunotoxins are described in, for example, Wayne et al. (2016) Blood, 123: 2470-2477, and include, for example, diphtheria toxin A, Ricin-dgA, Pseudomonas exotoxin A, Glonin, Liposomes, Particles or indeed any toxin delivery.

In one embodiment, the CD83 binding protein is an immunoconjugate comprising a diagnostic moiety. A diagnostic moiety is a compound, molecule or atom which is useful in the detection of binding of the antibody or antigen binding fragment to its target antigen. A diagnostic moiety can comprise a radionuclide or non-radionuclide, a contrast agent (such as for magnetic resonance imaging, computed tomography or ultrasound). Diagnostic moieties include, for example, radioisotopes, dyes (such as with the biotin-streptavidin complex), contrast agents, fluorescent compounds or molecules and enhancing agents (e.g., paramagnetic ions) for magnetic resonance imaging (MRI) or positron emission tomography (PET) scanning. In one embodiment, the diagnostic moieties are selected from the group consisting of radioisotopes, enhancing agents for use in magnetic resonance imaging, and fluorescent compounds. In order to load an antibody component with radioactive metals or paramagnetic ions, it may be necessary to react it with a reagent having a long tail to which are attached a multiplicity of chelating groups for binding the ions. Such a tail can be a polymer such as a polylysine, polysaccharide, or other derivatized or derivatizable chain having pendant groups to which can be bound chelating groups such as, e.g., ethylenediaminetetraacetic acid (EDTA), diethylenetriaminepentaacetic acid (DTPA), DOTA, NOTA, NETA, porphyrins, polyamines, crown ethers, bis-thiosemicarbazones, polyoximes, and like groups known to be useful for this purpose. Chelates are coupled to the antibodies using standard chemistries. The chelate is normally linked to the antibody by a group which enables formation of a bond to the molecule with minimal loss of immunoreactivity and minimal aggregation and/or internal cross-linking.

Methods for conjugating therapeutic and diagnostic moieties to an antibody or antigen binding fragment are known in the art.

The CD83 binding proteins described herein are typically formulated as a pharmaceutical composition for administration to the subject. Typically, the pharmaceutical composition comprises a CD83 binding protein formulated with a pharmaceutically acceptable carrier. A “pharmaceutically acceptable carrier” means that it is compatible with the other ingredients of the composition and is not deleterious to a subject. The compositions may contain other therapeutic agents as described below, and may be formulated, for example, by employing conventional liquid vehicles or diluents, as well as pharmaceutical additives of a type appropriate to the mode of desired administration (for example, excipients, binders, preservatives, stabilizers, flavours, etc.) according to techniques such as those well known in the art of pharmaceutical formulation (See, for example, Remington: The Science and Practice of Pharmacy, 21st Ed., 2005, Lippincott Williams & Wilkins).

Pharmaceutical compositions comprising the CD83 binding protein are typically in the form of a sterile injectable aqueous suspension. This suspension may be formulated according to the known art and contain the active materials in admixture with excipients suitable for the manufacture of aqueous suspensions. Such excipients may include suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectable formulations.

The pharmaceutical composition may be administered by any suitable means, typically, parenterally, such as by subcutaneous, intravenous, intramuscular, intra(trans)dermal, or intracisternal injection or infusion techniques (e.g., as sterile injectable aqueous solutions or suspensions); in dosage unit formulations containing non-toxic, pharmaceutically acceptable vehicles or diluents. The CD83 binding protein may, for example, be administered in a form suitable for immediate release or extended release. Immediate release or extended release may be achieved by the use of suitable pharmaceutical compositions comprising the compounds, or, particularly in the case of extended release, by the use of devices such as subcutaneous implants or osmotic pumps.

The pharmaceutical compositions for administration to the subject may conveniently be presented in dosage unit form and may be prepared by any of the methods well known in the art of pharmacy. In general, the pharmaceutical compositions are prepared by uniformly and intimately bringing the compound into association with a liquid carrier. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Generally, the term “treating” means affecting a subject, tissue or cell to obtain a desired pharmacological and/or physiological effect and include: (a) preventing the disease from occurring in a subject that may be predisposed to the disease, but has not yet been diagnosed as having it; (b) inhibiting the disease, i.e., arresting its development; or (c) relieving or ameliorating the effects of the disease, i.e., cause regression of the effects of the disease. In one embodiment, treatment achieves the result of reducing the number of malignant lymphocytes in the recipient subject.

The term “subject” refers to any animal having a disease which requires treatment by the present method. In addition to primates, such as humans, a variety of other mammals can be treated using the methods of the present invention. For instance, mammals including, but not limited to, cows, sheep, goats, horses, dogs, cats, guinea pigs, rats or other bovine, ovine, equine, canine, feline, rodent or murine species can be treated.

The term “effective amount” refers to the amount of the CD83 binding protein that will elicit the biological or medical response of a tissue, system, animal or human that is being sought by the researcher, veterinarian, medical doctor or other clinician.

In the treatment or prevention of lymphoma, an appropriate dosage level will generally be about 0.01 to 50 mg per kg patient body weight per dose. Preferably, the dosage level will be about 0.1 to about 25 mg/kg per dose; more preferably about 0.5 to about 10 mg/kg per dose. A suitable dosage level may be about 0.01 to 25 mg/kg per dose, about 0.05 to 10 mg/kg per dose, or about 0.1 to 5 mg/kg per dose. Within this range the dosage may be 0.05 to 0.5, 0.5 to 5 or 5 to 5 mg/kg per dose. Dosage may be administered once or multiple times. It will be understood that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In some examples, a dose escalation regime is used, in which a CD83 binding protein or other active ingredient is initially administered at a lower dose than used in subsequent doses. This dosage regime is useful in the case of subject's initially suffering adverse events.

In the case of a subject that is not adequately responding to treatment, multiple doses in a week may be administered. Alternatively, or in addition, increasing doses may be administered.

One or more CD83 binding proteins can be administered to a subject by an appropriate route, either alone or in combination with (before, simultaneous with, or after) another drug or agent. For example, the CD83 binding protein of the present disclosure can be administered in combination with, for example, one or more agents, such as one or more chemotherapeutic agents typically used for the treatment of lymphoma. Examples of chemotherapeutic agents suitable for the treatment of lymphoma include doxorubicin, bleomycin, vinblastine, decarbazine, etoposide, cyclophosphamide, vincristine, procarbazine, carmustine, etoposide, cytarabine, melphalan, chlorambucil, gemcitabibe, cisplatin, or combinations thereof. Chemotherapeutic agent combinations for treatment of lymphoma include ABVD (doxorubicin, bleomycin, vinblastine and dacarbazine), ChlVPP (chlorambucil, vinblastine, procarbazine and prednisone), ESHAP (etoposide, methylprednisolone, cytarabine, and cisplatin), BEAM (carmustine, etoposide, cytarabine, and melphalan), BEACOPP (bleomycin, etoposide, doxorubicin, cyclophosphamide, vincristine, procarbazine, and prednisolone).

In some embodiments, the CD83 binding protein may be administered in combination with one or more other binding proteins which may be effective for treatment of lymphoma. For example, the CD83 binding protein may be administered in combination with (before, simultaneously with, or after) with a PD-1 and/or PD-L1 binding protein, such as an anti-PD1 and/or anti-PD-L1 antibody. Examples of anti-PD1 or anti-PD-L1 antibodies are known in the art and include, for example, Nivolumab (Bristol-Myers Squibb), Pembrolizumab (Merck) and Atezolizumab (Roche).

### Diagnosing and Assessing

The CD83 binding protein may be used to diagnose or assess lymphoma.

In one aspect, the invention provides a method of diagnosing lymphoma in a subject, comprising determining the level of sCD83 in serum of the subject. The levels of sCD83 in serum of subjects suffering from lymphoma are elevated relative to the level of CD83 in subjects not suffering from lymphoma.

Another aspect provides a method of diagnosing, or assessing the severity or stage of lymphoma, comprising determining the level of sCD83 in serum of the subject and comparing the level of sCD83 in serum of the subject relative to the level of sCD83 in a subject not suffering from lymphoma, or suffering from lymphoma of known severity.

A further aspect provides a method of determining whether a subject is responding to treatment for lymphoma, comprising determining the level of sCD83 in serum of the subject before, during and/or after treatment, and comparing the level of sCD83 during and/or after treatment with the level of sCD83 before treatment, wherein the subject is responding to treatment when the level of sCD83 during and/or after treatment is reduced relative to the level of sCD83 before treatment.

As described in the Examples:
CD83 antibody binds tumour cells in HL lymph node biopsy samples;
serum of HL patients contain secreted CD83 (sCD83); and
the levels of sCD83 in patient's serum corresponds to the clinical response.

The inventors have found that the level of secreted CD83 in serum of patients correlates with the severity of lymphoma. As described in the Examples, subjects suffering from Hodgkin's lymphoma exhibit elevated levels of sCD83 compared to subjects non-suffering from lymphoma. Moreover, subjects suffering from Hodgkin's lymphoma have higher levels of sCD83 in their serum prior to chemotherapy treatment to reduce the lymphoma, as compared to sCD83 serum levels after treatment, indicating that a reduction in serum sCD83 correlates with disease severity.

The following assays can be performed with a CD83 binding protein of the disclosure, for example, a CD83 binding protein conjugated to a detectable label as discussed herein. Detection of CD83 with an assay described herein is useful for diagnosing or prognosing a condition.

An immunoassay is an exemplary assay format for diagnosing a condition in a subject or detecting CD83 in a sample. The present disclosure contemplates any form of immunoassay, including Western blotting, enzyme-linked immunosorbent assay (ELISA), fluorescence-linked immunosorbent assay (FLISA), competition assay, radioimmunoassay, lateral flow immunoassay, flow-through immunoassay, electro chemiluminescent assay, nephelometric-based assays, turbidometric-based assay, and fluorescence activated cell sorting (FACS)-based assays.

One form of a suitable immunoassay is, for example, an ELISA.

In one form, such an assay involves immobilizing a CD83 binding protein onto a solid matrix, such as, for example a polystyrene or polycarbonate microwell or dipstick, a membrane, or a glass support (e.g., a glass slide). A test sample is then brought into direct contact with the CD83 binding protein and CD83 in the sample is bound or captured. Following washing to remove any unbound protein in the sample, a protein that binds to CD83 at a distinct epitope is brought into direct contact with the captured CD83. This detector protein is generally labeled with a detectable reporter molecule, such as, for example, an enzyme (e.g. horseradish peroxidase (HRP)), alkaline phosphatase (AP) or (3-galactosidase) in the case of an ELISA. Alternatively, a second labeled protein can be used that binds to the detector protein. Following washing to remove any unbound protein the detectable reporter molecule is detected by the addition of a substrate in the case of an ELISA, such as, for example, hydrogen peroxide, TMB, or toluidine, or 5-bromo-4-chloro-3-indol-beta-D-galactopyranoside (x-gal). Of course, the immobilized (capture) protein and the detector protein may be used in the opposite manner.

The level of the antigen in the sample is then determined using a standard curve that has been produced using known quantities of the marker or by comparison to a control sample.

The assays described above are readily modified to use chemiluminescence or electrochemiluminescence as the basis for detection.

As will be apparent to the skilled artisan, other detection methods based on an immunosorbent assay are useful in the performance of the present disclosure. For example, an immunosorbent method based on the description supra using a radiolabel for detection, or a gold label (e.g., colloidal gold) for detection, or a liposome, for example, encapsulating NAD+ for detection or an acridinium linked immunosorbent assay. In some examples of the disclosure, the level of CD83 is determined using a surface plasmon resonance detector or bioluminometry (e.g., BIAcore™, GE Healthcare, Piscataway, N.J.), a flow through device, for example, as described in U.S. Pat. No. 7,205,159, a micro- or nano-immunoassay device (e.g., as described in U.S. Pat. No. 7,271,007), a lateral flow device (e.g., as described in US20040228761 or US20040265926), a fluorescence polarization immunoassay (FPIA e.g., as described in U.S. Pat. No. 4,593,089 or 4,751,190), or an immunoturbidimetric assay (e.g., as described in U.S. Pat. No. 5,571,728 or 6,248,597).

The method of diagnosing or assessing lymphoma may further comprise the step of treating the lymphoma. In one embodiment, the lymphoma is treated using the methods of treating lymphoma described herein.

Also disclosed herein is a kit comprising the CD83 binding protein described herein, typically comprising instructions for the treatment or diagnosis of lymphoma. In one embodiment, a kit comprises the CD83 binding protein, in one or more containers. In another embodiment, the kit comprises the CD83 binding protein described herein, in one or more containers, and one or more other therapeutic agents useful for the treatment of lymphoma. In another embodiment, the kit comprises the CD83 binding protein described herein, in one or more containers, and one or more other diagnostic moieties.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Throughout this specification, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

Each example of the present disclosure described herein is to be applied mutatis mutandis to each and every other example unless specifically stated otherwise.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

### Examples

### Materials and Methods

### Lymphoma Tissue Section and Plasma Samples

Archival paraffin embedded lymph node biopsies obtained from 35 HL, 20 DLBCL and 21 MCL patients at initial diagnosis were analysed after approval by the Sydney Local Health District (SLHD) Human Research Ethics Committee (HREC), consistent with the Declaration of Helsinki. Thirty-five HL patients had histological diagnosis of nodular sclerosis, mixed cellularity, lymphocyte rich classic, unspecified classic or nodular lymphocyte predominant HL under WHO/REAL classification (Swerdlow et al. Blood 2016; 127(20): 2375-2390). Plasma samples collected from 6 HL and 3 DLBCL patients at diagnosis and during chemotherapy were approved by the SLHD HREC. Positron emission tomography (PET)-scan was performed after 2-3 cycles of treatment in HL patients. MCL cell line Mino (ATCC® CRL3000™) was purchased from ATCC. DLBCL line Karpass-1106p was purchased from Cellbank Australia.

### Human Blood Cell and Cell Line Culture

Venous blood was collected from healthy donors (HD) under approval of SLHD HREC. Human PBMC were isolated by centrifugation on Ficoll-Paque-PLUS (GE Healthcare). T cells were isolated from PBMC using EasySep Human T cell Isolation Kit (STEMCELL Technologies) according to the supplier's instructions. Cell lines used in this study were HL cell lines KM-H2, L428 and HDLM-2 (gift from Prof Volker Diehl, University of Cologne, Germany). The HL-60 cell line was obtained from the Christchurch Haematology Research Group. Complete RPMI medium containing 10% fetal calf serum, 2 mM glutaMAX™, 100 U/ml penicillin, 100 μg/ml streptomycin, 1 mM sodium pyruvate, 10 mM HEPES, 10 μM β-Mercaptoethanol (Thermo Fisher Scientific) was used for cell culture throughout experiments.

### Flow Cytometry

The following antibodies were used: CD3-Alexa Fluor (AF)700, CD4-Phycoerythrin (PE)-CF594, CD15-Violet (V)450, CD19-V450, CD20-V421, CD30-PE, CD40-PE-Cy7, CD279 (PD-1)-Brilliant Violet (BV)786, CD274 (PD-L1)-PE-Cy7 (all from BD Biosciences), CD25-BV421 and CD107-PE-Cy7 (Biolegend). Mouse anti-human CD83 monoclonal antibodies (mAbs), HB15a-Fluorescein Isothiocyanate (FITC) was obtained from Beckman and Coulter, and HB15e-FITC from BD Biosciences. 3C12C is a human IgG1 anti-human CD83 mAb selected from a phage display library and further engineered by light chain shuffling to improve affinity (described in WO2014/117220). Isotype control antibodies included mouse IgG1 Kappa-FITC, mouse IgG2b-FITC (BD Biosciences) and human IgG1 Kappa (Sigma Aldrich). Data were collected on a Fortessa X20 flow cytometer (BD Biosciences) and analyzed with FlowJoV9&10 software (TreeStar).

### Immunofluorescence Staining

KM-H2, L428 or HDLM-2 cells (10⁵ cells) were cytospun onto lysine coated microscope slides. Cells were fixed and permeabilized with acetone at −20° C. overnight. This was followed by rehydration in PBS/1% BSA and blocking with 10% goat serum (Sigma Aldrich). Cells were stained with primary antibodies: HB15a (Beckman and Coulter), HB15e (STEMCELL Technologies) or 3C12C anti-CD83 antibodies, followed with goat anti-mouse IgG-AF647 (for HB15a, HB15e) or goat anti-human IgG-AF488 (for 3C12C) (Thermo Fisher Scientific). Nuclei were stained with 4′, 6-diamidino-2-phenylindole dihydrochloride (DAPI, Thermo Fisher Scientific). Cells were visualized using a laser scanning confocal microscope (Leica SP8) and composite images produced using Image J (National Institutes of Health).

### Immunohistochemistry

Immunohistochemical double staining was performed on 3 μm sections of formalin fixed paraffin embedded biopsy tissue of human lymph node from HL, MCL or BLBCL patients or non-human primates. The primary antibodies used were mouse anti-human CD20 (Dako), CD83 mAb (F5, Santa Cruz Biotechnology), CD30 (Dako) and staining was performed on a Leica Bond III Autostainer (Leica Biosystems) using a Bond Polymer Refine Detection kit for visualization with 3, 3′-diaminobenzidine (DAB). Images were taken with an Olympus BX51 microscopy with an Olympus PP71 camera using Olympus labSens software (Olympus).

### Trogocytosis Analysis

KM-H2 cells were cultured with purified CD3⁺ T cells from human PBMC for 4 hours at a ratio of 1:5. CD83 expression on T cells was analyzed by flow cytometry using HB15a mAb. For fluorescent imaging, KM-H2 cells were labelled with CellVue Claret Far Red Fluorescent Cell Linker Kits (Sigma-Aldrich) and co-cultured with CD3⁺ T cells for 4 hours at ratio of 5:1. Cells were then stained with biotinylated mouse anti-human CD3 mAb (BD Bioscience) and Strepdavidin-AF488 (Thermo Fisher Scientific). In some experiments, 0.4 μm transwell insert (Corning) were used to separate T cells from KM-H2 cells during culture. CD83 expression on T cells was analyzed by flow cytometry after 4 hours of culture.

### T Cell Proliferation Analysis

T cells isolated from human PBMC were labelled with 5 nM Carboxyfluorescein N-hydroxysuccinimidyl ester (CFSE; Sigma-Aldrich) and stimulated with anti-CD2/CD3/CD28 T cell activation/expansion kit (Miltenyi Biotec) in the presence of supernatant from KM-H2 cells for 5 days. Cells were analysed by flow cytometry on a Fortessa (BD Bioscience). The proliferation Index (PI) and Division Index (DI) were analysed with FlowJo V9 (TreeStar).

sCD83 and IL-10 Analysis

Human sCD83 in the culture supernatant of KM-H2, L428 or the serum of HL patient was analyzed by ELISA (Sino Biological Inc) according to the manufacturer's instructions, which has a detection limit of 3.9 pg/ml. Briefly, a 96 well plate was coated overnight with the supplied CD83 capture mAb. Culture supernatant, patient plasma or a recombinant CD83-Fc standard (from the sCD83 ELISA kit) was incubated for 2 hours, and sCD83 was detected using a mouse anti-human CD83-HRP and tetramethylbenzidine (Sigma-Aldrich) substrate solution, which was read at 450 nM on a microplate reader (PerkinElmer). IL-10 levels in cell line supernatants were analyzed by cytometric bead array (CBA; BD Bioscience).

### Antibody Dependent Cell Cytotoxicity (ADCC) Assays

KM-H2, L428 or HDLM-2 cells were used as target cells and labeled with 2504 Calcein-AM (Life Technologies) at 37° C. for 30 min and human PBMC were used as effector cells. Effector cells and target cells (5×10³ per well) at E:T ratio of 25:1 were co-incubated in triplicate for 3 hours at 37° C. with 3C12C at various concentrations or control anti-CD20 antibody, Rituximab (Roche). Supernatants were collected to measure released calcein (excitation 485 nm, emission 538 nm) using an ELISA Reader (Perkin Elmer). The percentage of specific cytolysis was calculated using the formula: percentage specific lysis=[E/T (sample)−E/T (spontaneous)]/[T (total)−T (spontaneous)]×100, where T (spontaneous)=target only, E/T (spontaneous)=effector+target, T (total)=target+lysis.

3C12C Conjugation with Monomethyl Auristatin E (3C12C-MMAE) and Cytotoxicity on CD83⁺ Cell Lines To produce 3C12C-MMAE, a lysosomal cathepsin B cleavable, self-emolative dipeptide (ValCit) maleimide linker was prepared from auristatin E for conjugation to partially reduced 3C12C using a similar method to Brentuximab Vedotin²⁸. The cytotoxic activity of 3C12C-MMAE on HL cells were analysed in vitro by incubating various concentrations of conjugate with CD83⁺ lymphoma cells or CD83⁻ (for specificity) HL-60 cell lines for 3 days. Viability was assessed by 7-amino-actinomycin D (7AAD, Thermo Fisher Scientific) staining using flow cytometry.

### PCR Analysis

RNA was extracted with TRIzol (Life Technologies) and cDNA was transcribed from 100 ng RNA using SuperScript® III First-Strand Synthesis kit and random hexamers primer (Thermo Fisher Scientific) following the manufacturer's protocol. cDNA from the specified immune populations were amplified by PCR using human CD83 exon 2 forward primer 5′-AGGTTCCCTACACGGTCTCC-3′ and exon 5 reverse primer 5′-AAGATACTCTGTAGCCGTGCAAAC-3′. Primers to the GAPDH housekeeping gene 5′-ATGGGGAAGGTGAAGGTCGGA-3′ (forward) and 5′-AGGGGCCATCCACAGTCTTCTG-3′ (reverse) were used as an endogenous control. Amplified fragments were separated on 2% agarose (Thermo Fisher Scientific) gel.

### 3C12C Trials in Non-Human Primates

The SLHD Animal Research Ethics Committee approved the study of 5 non-human primates (Papio Hamadryas baboon), which received intravenous human-IgG (Intragam, CSL) (10 mg/kg) or 3C12C mAb (1, 5, 10, 10 mg/kg) at days 0, 7, 14 and 21. Blood counts were performed using a CELL-DYN Sapphire automated blood counter (Abbott). PBMC were analyzed for immune cell populations including DC, T and B cells on a Fortessa X20 flow cytometer (BD Biosciences). Liver and kidney function were assessed by measuring ALP, AST & creatinine (Cr) in serum samples collected weekly until day 56 using the Cobas 8000 (Roche). Lymph nodes were taken from 3C12C (10 mg/kg) or human IgG (10 mg/kg) treated animals at day 28 for immunohistological staining.

### Statistical Analysis

Statistical analyses were performed using Prism 6.0 (GraphPad Software). Standard error of the mean is shown unless otherwise stated. A Mann-Whitney t-test or one-way ANOVA test with Greenhouse-Geisser correction for multiple comparisons were used as described. Differences with p<0.05 were considered significant.

### Results

### 1. CD83 is Expressed on HL Cell Lines and HRS in Lymph Node Biopsies of HL Patients

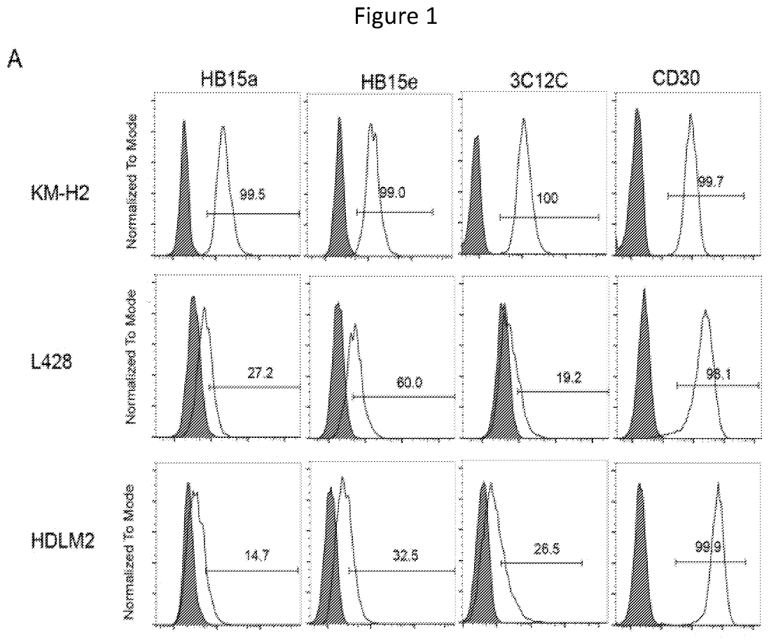

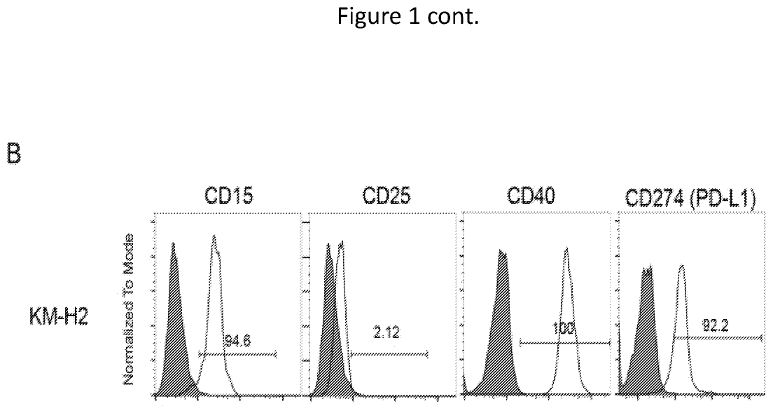

Expression of CD83 was analyzed using the mouse anti-human antibodies HB15a, HB15e and potential therapeutic human anti-human CD83 antibody 3C12C. Expression of CD83 was analyzed by flow cytometry on KM-H2, L428 and HDLM-2 lymphoma cell lines, which were stained with HB15a-FITC, HB15e-FITC or 3C12C-FITC anti-CD83 mAbs, respectively KM-H2 cells expressed the most cell surface CD83 stained with all antibodies, whilst the L428 and HDLM-2 lines expressed less CD83. All three lines expressed CD30 (FIG. 1A). CD15, CD25, CD40 and CD274 (PD-L1) were expressed on KM-H2 cells (FIG. 1B). This data was confirmed by confocal CD83 staining on KM-H2 cells and detection of CD83 mRNA transcripts by RT-PCR in the three HL lines (FIG. 8).

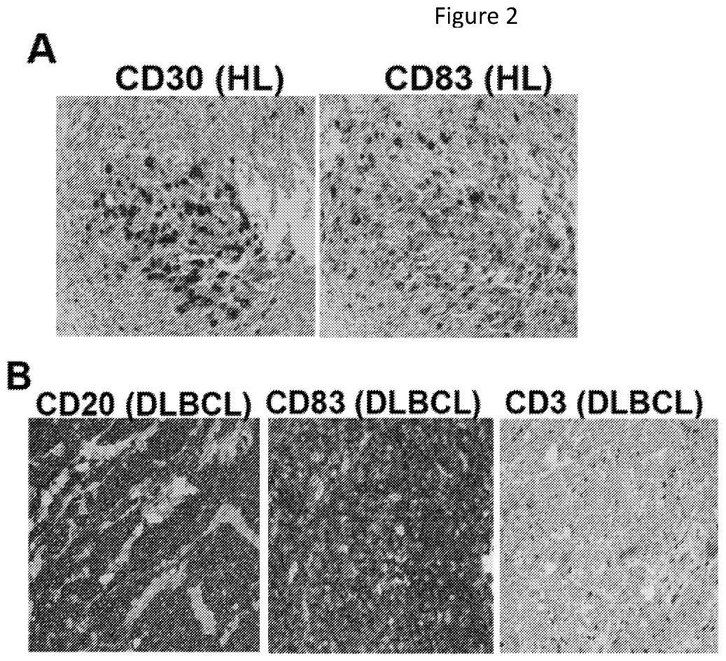

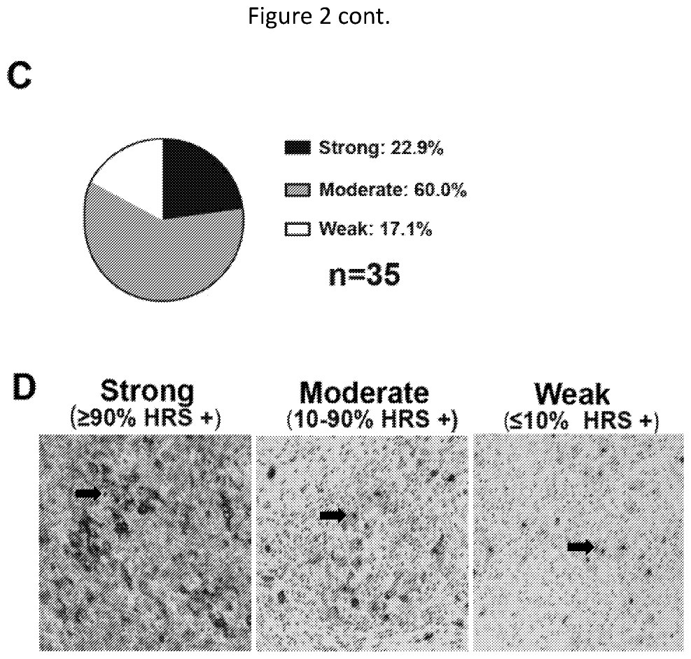

Next, CD83 expression was analyzed on the paraffin embedded lymph node biopsies of 35 HL patients. The HRS cells were identified as CD30⁺ (FIG. 2A). Of note, 8/35 (22.9%) biopsies of HL patients expressed high levels of CD83 on the HRS cells (>90% positive), 21/26 (60%) expressed moderate levels (10-90% positive) and 6/35 (17.1%) expressed low levels of CD83 (<10% positive) (FIG. 2C). The subtype analysis showed that 81% of HRS cells in nodular sclerosis (NS) HL were CD83 high or moderate and 85.7% were CD83 high or moderate in mixed cellularity (MC) HL. Most (90%) of stage I-II HL were CD83 high or moderate and 61.5% HL in stage III-IV were CD83 high or moderate.

CD83 expression was also analyzed on the paraffin embedded lymph node biopsies of MCL and DLBCL patients. The biopsies from DLBCL patients showed expression of high levels of CD83 and CD20, and low levels of CD3 (FIG. 2B). The biopsies from mantle cell lymphoma patients also showed expression of high levels of CD83 (FIG. 13).

CD83 is Trogocytosed from HL Cells to T Cells.

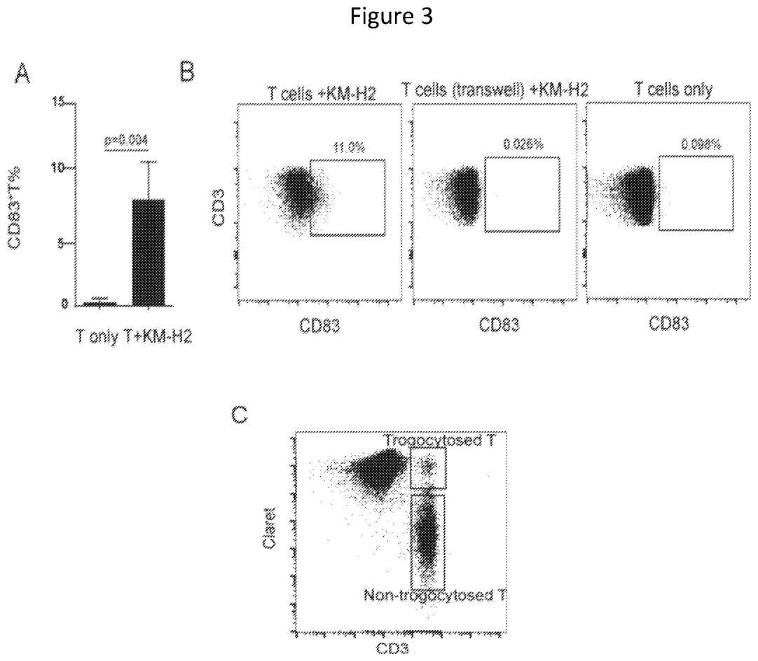

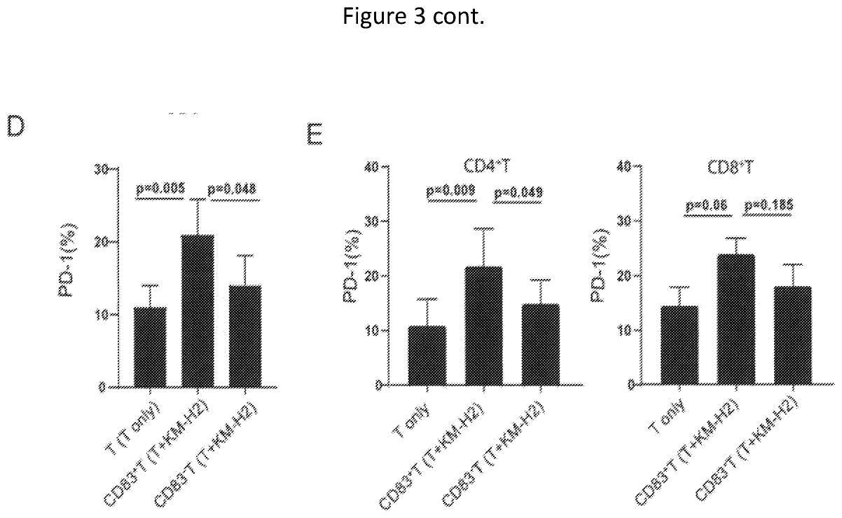

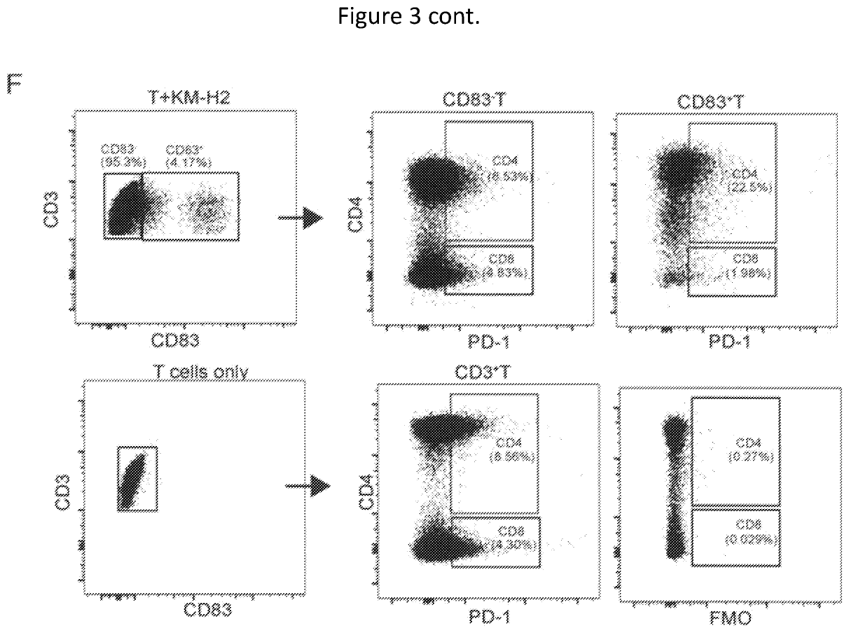

We found previously that CD83 was able to transfer from the membrane of DC to T cells via trogocytosis (Ju X et al. Journal of immunology 2016; 197(12): 4613-4625). Similar trogocytosis was observed to occur between HL cell lines and T cells. When these two cell types were co-cultured for 4 hours, CD83 surface expression was increased on T cells to between 5-12% (FIG. 3A; p=0.004), whereas no CD83 was detected on T cell in the absence of KM-H2 cells. Furthermore, separating the T and KM-H2 cells during culture by a 0.4 μm transwell filter prevented trogocytosis (FIG. 3B). To confirm the trogocytosis involved membrane transfer, KM-H2 cells were labelled with fluorescent dye (CellVue Claret) and co-cultured with CD3⁺ T cells. Cell membrane transfer from KM-H2 cells to T cells was confirmed by flow cytometry (FIG. 3C) and confocal microscopy. No differences occurred in the CD4⁺ and CD8⁺ T cell ratio during the co-culture of KMH2 and T cells within 4 hours. However, the CD83⁺ T cells expressed significantly higher levels of PD-1 than CD83⁻ T cells (p=0.048) and T cells cultured without KM-H2 (p=0.005) (FIG. 3D). The increase in PD-1 was significantly higher on the trogocytosed CD83⁺CD4⁺ T cells than non-trogocytosed CD83⁻ T cells (p=0.049). In contrast, no difference in PD-1 expression was seen between the CD83⁺ and CD83⁻ CD8⁺ T cells, (p=0.185) although both KM-H2 co-cultured CD4⁺ and CD8⁺ T cells had higher PD-1 expression than T cells cultured alone (FIG. 3E, F). The CD83⁺CD4⁺ T cells had the same proportion of Treg as non-trogocytosed CD4⁺ T cells (FIG. 9).

Supernatant from HL Cell Lines Inhibit T Cell Proliferation.

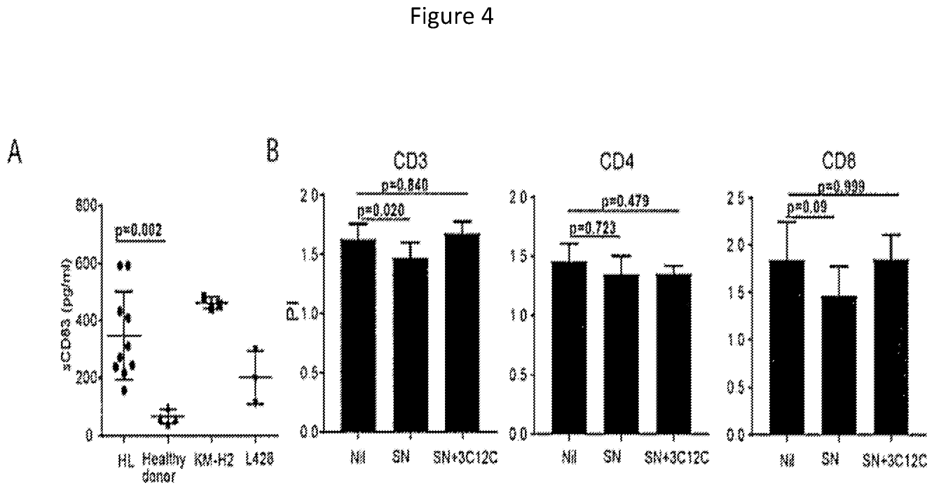

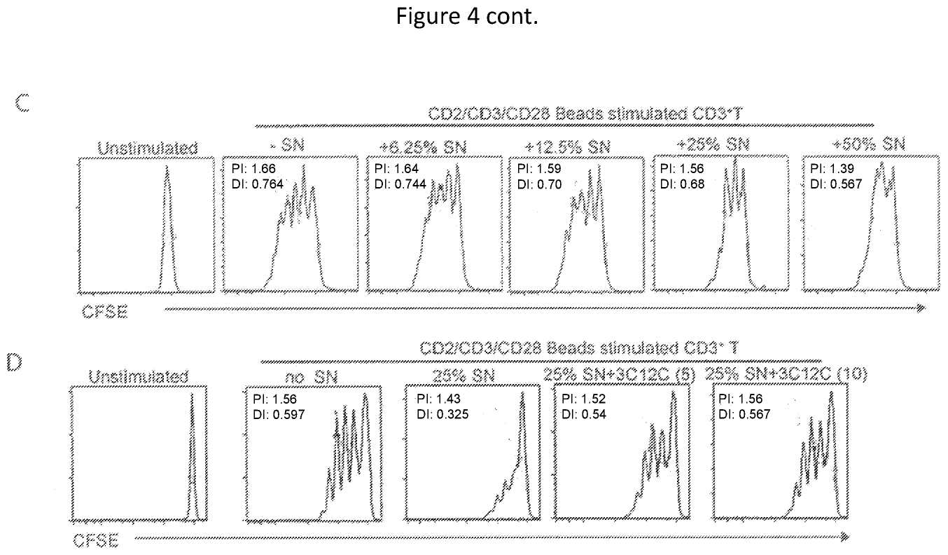

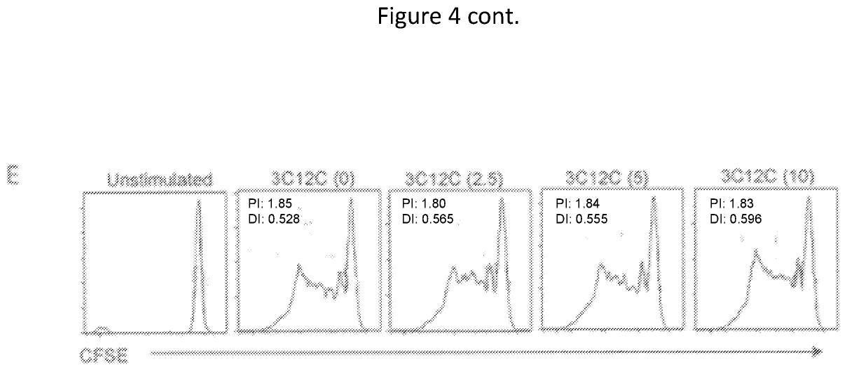

High levels of sCD83 were found in the supernatant of KM-H2 (460.6±11.8 pg/ml) and L428 (200.8±53.2 pg/ml), consistent with their high level of surface CD83 (FIG. 4A). HL patients had significantly higher serum sCD83 (360.5±54.82 pg/ml, n=10) at diagnosis than healthy donor (HD) (52.6±9.5 pg/ml. FIG. 4A). Interestingly, very low IL-10 levels were present in the supernatants of all three HL cell lines (FIG. 10).

We then tested the effect of KM-H2 cell supernatant on T cell function. KM-H2 supernatant containing sCD83 inhibited T cell proliferation in a dose-dependent manner (FIG. 4B-4E). Only proliferation of CD8⁺ T cells seemed inhibited by KM-H2 supernatant (p=0.09), and not CD4⁺ T cell proliferation (p=0.732) (FIG. 4B). Administration of the anti-CD83 antibody, 3C12C, partially abolished the inhibitory effect of KM-H2 supernatant (Figure. 4B, 4C and 4D). 3C12C alone had no effect on T cell proliferation (FIG. 4E).

HL Patient Serum sCD83 Declined to Normal Levels Correlated with a Complete or Partial Response by PET-Scan

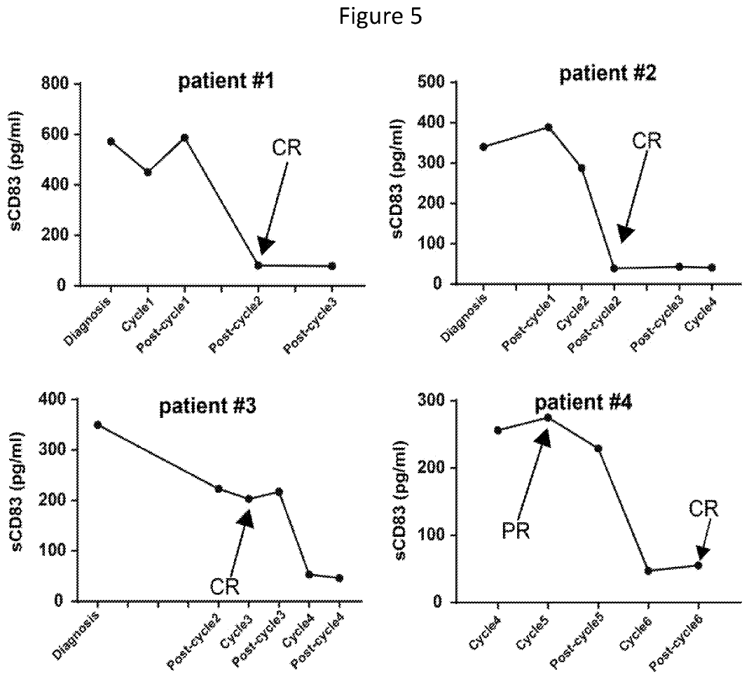

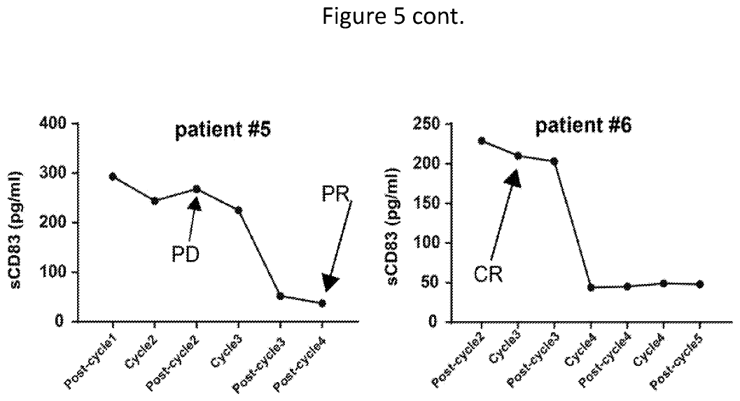

We monitored changes in circulating sCD83 in HL patients during sequential chemotherapy for six patients. All patients received 3-6 cycles of chemotherapy and five achieved a complete response (CR) and one patient a partial response (PR) by PET-scan (FIG. 5). Serum sCD83 decreased, returning to normal levels when the patients had a CR to chemotherapy documented by PET-scan in patients #1 and 2. In patient #3 and 6, the serum sCD83 level was still elevated when the PET-scan showed CR but normalized after one further cycle of chemotherapy. Patient #4 showed a PR prior to cycle 5 by PET-scan, however the serum sCD83 level only started to decrease during cycle #5 reaching a normal range in cycle 6, coinciding with CR. PET-scans in patient #5 showed progressive disease (PD) after cycle 2, but a PR after another 2 cycles of chemotherapy, when the corresponding sCD83 reduced to normal.

### 3C12C Kills HL Cell Lines Via ADCC

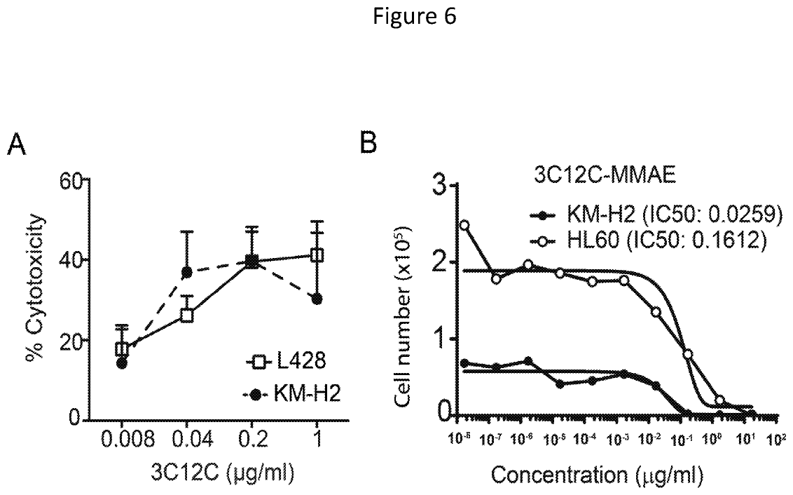

The ADCC activity of the anti-CD83 mAb, 3C12C, was tested on the three HL lines: KM-H2, L428 and HDLM-2. Whilst 3C12C killed KM-H2 and L428 efficiently, HDLM-2 was relatively resistant to it (FIG. 6A). To investigate further potential therapeutic applications, we generated a 3C12C toxin-conjugate (3C12C-MMAE). In vitro, 3C12C-MMAE killed CD83⁺ KM-H2 cells more efficiently than CD83⁻ HL-60 cells (FIG. 6B).

### Administration of 3C12C is Safe in Mouse and Non-Human Primate (NHP)

We performed dose-escalation studies of 3C12C in non-human primates. Five baboons were injected intravenously with 3C12C (1, 5, 10 mg/kg on d0, 7, 14, and 21). No adverse clinical events were recorded during follow up for 84 days post injection. We assessed blood counts and biochemistry weekly and monitored different immune cell population by flow cytometry or immune histology. Administration of 3C12C did not affect blood cell counts (WBC, RBC, and platelets), liver (ALP and AST) or kidney (Creatinine) function (FIG. 12). The total T cell number, ratio of CD4⁺/CD8⁺ T cells all remained normal up to day 84 (data not shown). However, there was evidence of 3C12C efficacy in that other CD83⁺ target cells (activated DC and activated B cells) were reduced. Intravenous administration of 3C12C to mice resulted in reductions in blood and lymph node B cells as determined by flow cytometry (FIG. 7A). In addition, B cell areas in lymph node were reduced in a 3C12C treated animal (10 mg/kg) compared to a control human IgG animal (10 mg/kg; FIG. 7B).

### MCL and FL CD83 Staining

Staining with anti-CD83 antibody of MCL shows strong diffuse membranous and cytoplasmic staining. In addition, there is strong punctate staining, just like the DLBCL (FIG. 13 [MCL]). 52.2% MCL biopsy samples expressed high or middle level of CD83 (n=21). The FL shows anti-CD83 staining of the reactive B cells around and within the follicles. There is no diffuse staining of the FL (FIG. 13 [FL]).

### 3C12C-MMAE Kill DLBCL and MCL Cell Lines

To determine the effect of 3C12C-MMAE conjugates on DLBCL and MCL cells lines, the DLBCL line KARPASS-1106P or MCL line Mino cells were incubated with different concentrations of 3C12C-MMAE for 72 hours, the viable cells were counted by flow cytometry. KM-K2 cells were used as a control. A plot of the viable cells number with increasing antibody conjugate concentration for each of cell lines Mino, KM-H2 and Karlasss, together with the half maximal inhibitory concentration (IC₅₀) calculated, is shown in FIG. 14. Similar to HL line KM-H2, DLBCL and MCL lines were effectively killed by 3C12C-MMAE after 72 hours of culture (FIG. 14).

### SUMMARY

High expression of CD83 on HL cell lines and primary HL, DLBCL and MCL tissues indicates CD83 is a good therapeutic target.
HL tumour cells express CD83 and some surrounding T cells can acquire surface CD83 molecules from tumor cells. The 80% of CD83⁺ T cells were CD4+ T cells with the high expression of co-inhibitory molecule e.g. PD-1. The infiltrating T lymphocytes in HL patients are hyporesponsive to antigen. PD-1 and PD-1 ligand interaction contributes to the immunosuppressive microenvironment of Hodgkin lymphoma. CD83 transferred from KM-H2 to T cells in vitro is consistent with the finding that expression of CD83 on lymphocytes of LN biopsy samples, especially in CD83 high expression patients. Such CD83+ T cells might become exhaustive or apoptotic (as PD-1 high) this may be another mechanism that KM-H2 cells escape immune-surveillance via CD83. This indicates CD83 target therapy combined with PD-1 inhibitors will probably further enhance the clinical response.
Supernatants (SN) of HL cells inhibit T cell proliferation (SN of KM-H2 does not induce Treg, data not shown), HL cells secrete high sCD83 into SN and sCD83 were detected higher in HL patient serum than healthy donors; 3C12C partially abolishes such inhibition by binding the sCD83. sCD83 from SN plays a major role in such inhibitory effect but not IL-10. 3C12C had no effect on the inhibitory function of Treg in vitro although 3C12C might induce transient Treg (from NHP trial data) which could be the indirect effect of Treg induction from non-activated DC, 3C12C delete activated DC in vitro and in vivo. In contrast, the PD-1 inhibitor (Nivolumab) directly limits the Treg suppressive function of CD8+ T cells and increased the ratio of CD8/Treg and CD4 Teffs/Treg in mice receiving Nivolumab.
Other cytokines or soluble factors from SN might also contributes to the inhibitory effect e.g. sCD30. An 85 kDa soluble form of the CD30 molecule (sCD30) has been shown to be released by CD30+ cell in vitro and in vivo. Activated T cells especially CD4+ T cell also secreted sCD30.
sCD83 level in HL patients correlated with disease status and treatment response. Thus, sCD83 level could be a diagnostic and prognostic biomarker.
Anti-CD83 antibody, 3C12C, (and CD83mAb drug complex) kills HRS cells, DLBCL and MCL cells in vitro. In NHP trials, human anti CD83 mAb 3C12C is safe without side effect on blood cell count, liver and kidney function, the efficacy and safety profile make the CD83 antibody as another candidate of effective therapeutic antibodies for HL.

1. A method of treating lymphoma in a subject, comprising administering to the subject an effective amount of a CD83 binding protein.

2. The method of claim 1, wherein the lymphoma is HL.

3. The method of claim 1, wherein the lymphoma is NHL.

4. The method of claim 3, wherein the NHL is MCL.

5. The method of claim 3, wherein the NHL is DLBCL.

6. The method of claim 3, wherein the NHL is FL.

7. The method of claim 1, wherein the CD83 binding protein comprises an antigen binding domain which comprises:
(a) a heavy chain variable region (VH) which comprises:
(i) a sequence which is at least 90% identical to the amino acid sequence shown in SEQ ID NO:10; or
(ii) three complementarity determining regions (CDRs) of the amino acid sequence shown in SEQ ID NO:10; and
(b) a light chain variable region which comprises:
(i) a CDR1 sequence comprising the amino acid sequence of SEQ ID NO: 37, a CDR2 sequence comprising the amino acid sequence of SEQ ID NO: 38 and a CDR3 sequence comprising the amino acid sequence of SEQ ID NO: 39.

8. The method of claim 1, wherein the CD83 binding protein comprises an antigen binding domain which comprises:
(a) a heavy chain variable region which comprises a CDR1 sequence comprising the amino acid sequence of SEQ ID NO: 4, a CDR2 sequence comprising the amino acid sequence of SEQ ID NO: 5 and a CDR3 sequence comprising the amino acid sequence of SEQ ID NO: 6; and
(b) a light chain variable region which comprises a CDR1 sequence comprising the amino acid sequence of SEQ ID NO: 7, a CDR2 sequence comprising the amino acid sequence of SEQ ID NO: 8 and a CDR3 sequence comprising the amino acid sequence of SEQ ID NO: 9.

9. The method of claim 8, wherein the CD83 binding protein comprises an antigen binding domain which comprises a variable heavy chain comprising an amino acid sequence of SEQ ID NO: 10, and a variable light chain comprising an amino acid sequence of SEQ ID NO: 11.

10. The method of claim 1, wherein the CD83 binding protein comprises an antigen binding domain which comprises:
a V_{H} sequence as shown in SEQ ID NO:10 and a V_{L} sequence as shown in SEQ ID NO:12; or
(ii) a V_{H} sequence as shown in SEQ ID NO:10 and a V_{L} sequence as shown in SEQ ID NO:13; or
(iii) a V_{H} sequence as shown in SEQ ID NO:10 and a V_{L} sequence as shown in SEQ ID NO:14; or
(iv) a V_{H} sequence as shown in SEQ ID NO:10 and a V_{L} sequence as shown in SEQ ID NO:15; or
(v) a heavy chain sequence as shown in SEQ ID NO: 21 and a light chain sequence as shown in SEQ ID NO:16; or
(vi) a heavy chain sequence as shown in SEQ ID NO: 21 and a light chain sequence as shown in SEQ ID NO:17; or
(vi) a heavy chain sequence as shown in SEQ ID NO:21 and a light chain sequence as shown in SEQ ID NO:18; or
(vii) a heavy chain sequence as shown in SEQ ID NO:21 and a light chain sequence as shown in SEQ ID NO:19; or
(viii) a heavy chain sequence as shown in SEQ ID NO:21 and a light chain sequence as shown in SEQ ID NO:20; or
(ix) a V_{H} sequence as shown in SEQ ID NO:22 and a V_{L} sequence as shown in SEQ ID NO:23; or
(x) a V_{H} sequence as shown in SEQ ID NO:22 and a V_{L} sequence as shown in SEQ ID NO:24; or
(xi) a V_{H} sequence as shown in SEQ ID NO:22 and a V_{L} sequence as shown in SEQ ID NO:25;
(xii) a V_{H} sequence as shown in SEQ ID NO:22 and a V_{L} sequence as shown in SEQ ID NO:26;
(xii) a V_{H} sequence as shown in SEQ ID NO:22 and a V_{L} sequence as shown in SEQ ID NO:27;
(xiv) a V_{H} sequence as shown in SEQ ID NO:22 and a V_{L} sequence as shown in SEQ ID NO: 28;
(xv) a V_{H} sequence as shown in SEQ ID NO:22 and a V_{L} sequence as shown in SEQ ID NO: 29;
(xvi) a V_{H} sequence as shown in SEQ ID NO:22 and a V_{L} sequence as shown in SEQ ID NO: 30;
(xvii) a V_{H} sequence as shown in SEQ ID NO:22 and a V_{L} sequence as shown in SEQ ID NO: 31;
(xviii) a V_{H} sequence as shown in SEQ ID NO:22 and a V_{L} sequence as shown in SEQ ID NO: 32;
(xix) a V_{H} sequence as shown in SEQ ID NO:22 and a V_{L} sequence as shown in SEQ ID NO: 33; or
(xx) a V_{H} sequence as shown in SEQ ID NO:22 and a V_{L} sequence as shown in SEQ ID NO: 34; or
(xxi) a heavy chain sequence as shown in SEQ ID NO: 35 and a light chain sequence as shown in SEQ ID NO: 36.

11. The method of claim 10, wherein the CD83 binding protein is an antibody.

12. The method of claim 10, wherein the CD83 binding protein is a monoclonal antibody or antigen binding fragment thereof.

13. The method of claim 10, wherein the CD83 binding protein is selected from the group consisting of Fab, Fab′, F(ab′)₂, Fab₂, and scFv.

14. The method of claim 1, wherein the CD83 binding protein is conjugated to a moiety.

15. (canceled)

16. The method of claim 14, wherein the moiety is a therapeutic or diagnostic moiety.

17. A method of diagnosing, or assessing the severity or stage of lymphoma, comprising comparing the level of sCD83 in serum of the subject relative to the level of sCD83 in a subject not suffering from lymphoma, or suffering from lymphoma of known severity.

18. A method of determining whether a subject is responding to treatment for lymphoma, comprising determining the level of sCD83 in serum of the subject before, during and/or after treatment, and comparing the level of sCD83 during and/or after treatment with the level of sCD83 before treatment, wherein the subject is responding to treatment when the level of sCD83 during and/or after treatment is reduced relative to the level of sCD83 before treatment.

19. The method of claim 17, wherein the lymphoma is HL or NHL.

20. The method of claim 18, wherein the lymphoma is HL or NHL.

21. The method of claim 19, wherein the NHL is DLBCL or MCL.

22. The method of claim 20, wherein the NHL is DLBCL or MCL.

23. (canceled)


SEQUENCE LISTING (59 sequences)

SEQ ID NO: 1 - protein, 205 aa, human
MSRGLQLLLLSCAYSLAPATPEVKVACSEDVDLPCTAPWDPQVPYTVSWVKLLEGGEERMETPQEDHLRGQHYHQKGQNGSFDAPNERPYSLKIRNTTSCNSGTYRCTLQDPDGQRNLSGKVILRVTGCPAQRKEETFKKYRAEIVLLLALVIFYLTLIIFTCKFARLQSIFPDFSKAGMERAFLPVTSPNKHLGLVTPHKTELV

SEQ ID NO: 2 - protein, 204 aa, human
MSRGLQLLLLSCAYSLAPATPEVKVACSEDVDLPCTAPWDPQVPYTVSWVKLLEGGEERMETPQEDHLRGQHYHQKGQNGSFDAPNERPYSLKIRNTTSCNSGTYRCTLQDPDGQRNLSGKVILRVTGCPAQRKEETFKKYRAEIVLLLALVIFYLTLIIFTCFARLQSIFPDFSKAGMERAFLPVTSPNKHLGLVTPHKTELV

SEQ ID NO: 3 - protein, 146 aa, Human
METPQEDHLRGQHYHQKGQNGSFDAPNERPYSLKIRNTTSCNSGTYRCTLQDPDGQRNLSGKVILRVTGCPAQRKEETFKKYRAEIVLLLALVIFYLTLIIFTCKFARLQSIFPDFSKAGMERAFLPVTSPNKHLGLVTPHKTELV

SEQ ID NO: 4 - protein, 5 aa, Artificial
  CDR sequence
SYAMH

SEQ ID NO: 5 - protein, 10 aa, Artificial
  CDR sequence
VISYDGSNKY

SEQ ID NO: 6 - protein, 8 aa, Artificial
  CDR sequence
HYYYGMDV

SEQ ID NO: 7 - protein, 11 aa, Artificial
  CDR sequence
RASQGIRNYLA

SEQ ID NO: 8 - protein, 7 aa, Artificial
  CDR sequence
ATSTLQS

SEQ ID NO: 9 - protein, 9 aa, Artificial
  CDR sequence
QQVDRFPYT

SEQ ID NO: 10 - protein, 117 aa, Artificial
  heavy chain variable region
EVQLQESGGGVVQPGRSLRLSCAASGFTFSSYAMHWVRQAPGKGLEWVAVISYDGSNKYYADSVKGRFTISRDNSKNTLYLQMNSLRAEDTAMYYCARHYYYGMDVWGQGTTLTVSS

SEQ ID NO: 11 - protein, 107 aa, Artificial
  light chain variable region
EIVLTQSPSSLSASVGDRVTITCRASQGIRNYLAWYQQKPGKVPKLLIYATSTLQSGVPSRFSGSRSGTEFTLTISSLHPEDFATYYCQQVDRFPYTFGQGTKVELK

SEQ ID NO: 12 - protein, 107 aa, Artificial
  light chain variable region
EIVMTQSPSLLSASLGDRVTITCRASQGIKNYFAWYQQRPGKAPKLLIYATSNLQSGVPSRFSGSGSGTEFTLTISSLQPEDFATYYCQQLGAYPLTFGGGTKLELK

SEQ ID NO: 13 - protein, 107 aa, Artificial
  light chain variable region
EVVMTQSPSFLSASIGDRVSITCRTSQGISNHLAWYQQKPGKAPKLLIYAASSLQSGVPSRFSGSGSGTDFTLTISSLQPEDIATYYCQQVNSYPYTFGQGTRLELK

SEQ ID NO: 14 - protein, 107 aa, Artificial
  light chain variable region
EIQMTQSPSSLSASVGDRVTITCRASQGISSWLAWYQQKPGKAPKLLIYAASSLQSGVPSRFSGSGSGTEFTLTISSLQPDDFATYYCQKLSSYPYTFGGGTKVELK

SEQ ID NO: 15 - protein, 107 aa, Artificial
  light chain variable region
EIVLTQSPSSLSASVGDRVTITCRASQGISNYLAWYQQKPGKVPKLLIYAASTLQSGVPSRFSGSGSGTHFTLTISSLQPEDVATYYCQKCNSAPYTFGQGTKVELK

SEQ ID NO: 16 - protein, 214 aa, Artificial sequence
  ligh chain
EIVMTQSPSLLSASLGDRVTITCRASQGIKNYFAWYQQRPGKAPKLLIYATSNLQSGVPSRFSGSGSGTEFTLTISSLQPEDFATYYCQQLGAYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 214 aa, Artificial
  light chain
EVVMTQSPSFLSASIGDRVSITCRTSQGISNHLAWYQQKPGKAPKLLIYAASSLQSGVPSRFSGSGSGTDFTLTISSLQPEDIATYYCQQVNSYPYTFGQGTRLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 214 aa, Artificial
  light chain
EIVLTQSPSSLSASVGDRVTITCRASQGIRNYLAWYQQKPGKVPKLLIYATSTLQSGVPSRFSGSRSGTEFTLTISSLHPEDFATYYCQQVDRFPYTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 214 aa, Artificial
  light chain
EIQMTQSPSSLSASVGDRVTITCRASQGISSWLAWYQQKPGKAPKLLIYAASSLQSGVPSRFSGSGSGTEFTLTISSLQPDDFATYYCQKLSSYPYTFGGGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 214 aa, Artificial
  light chain
EIVLTQSPSSLSASVGDRVTITCRASQGISNYLAWYQQKPGKVPKLLIYAASTLQSGVPSRFSGSGSGTHFTLTISSLQPEDVATYYCQKCNSAPYTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, Artificial
  heavy chain
EVQLQESGGGVVQPGRSLRLSCAASGFTFSSYAMHWVRQAPGKGLEWVAVISYDGSNKYYADSVKGRFTISRDNSKNTLYLQMNSLRAEDTAMYYCARHYYYGMDVWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 221 aa, Artificial
  VH amino acid sequence of 1F7
EVQLVQSGGAVVQPGRSLRLSCAASGFTFSTYGMHWVRQAPGKGLEWVAAVSYDGSNKYYADFVKGRFTISRDNPKNTLYLQMNSLRADDTAVYYCARRGGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 23 - protein, 108 aa, Artificial
  ligh chain VL amin acid sequence of 1F7
LTQPPPASGTPGQRVTISCSGSSSNIGSNTVNWYQQLPGTAPKLLIYGNDQRPSGVPDRFSASKSGTSASLAISGLQSEDEAHYYCAAWDGSLNGGVIFGGGTKVTLG

SEQ ID NO: 24 - protein, 109 aa, ARtificial
  light chain VL amino acid sequence of hFab4.1
VTQPPSASGTPGQRVTISCSGSSSNIGTNPVNWYQQLPGTAPKLLIYTTDQRPSGVPDRFSGSKSGTSASLAISGLQSEDEADYYCAAWDDSLSGLYVFGTGTKVTVLG

SEQ ID NO: 25 - protein, 109 aa, Artificial
  light chain VL amino acid sequence of hFab4.2
MTHTPLSLSVTPGQPASISCKSSQSLLHSDGKTYLYWYLQRPGQSPQPLIYEVSNRFSGVPDRFSGSGSGTDFTLKISRVEAEDVGVYYCMQSLQLWTFGQGTKVEIKR

SEQ ID NO: 26 - protein, 110 aa, Artificial
  light chain VL amino acid sequence of hFab4.3
MTQSPLSLPVTLGQPASISCRSSQSLIHSDGNTYLDWFQQRPGQSPRRLIYKVSNRDSGVPDRFSGSGSGTDFTLRISRVEAEDIGVYYCMQATHWPRTFGQGTKVEIKR

SEQ ID NO: 27 - protein, 110 aa, Artificial
  light chain VL amino acid sequence of hFab4.4
MTQSPLSLPVTLGQPASISCRSSQSLVDSAGNTFLHWFHQRPGQSPRRLIYKVSNRDSGVPDRFSGSGSGTDFTLKISRVEAEDVGVYYCMQGTHWPRTFGQGTKVEIKR

SEQ ID NO: 28 - protein, 110 aa, Artificial
  light chain VL amino acid sequence of hFab4.5
LTQSPLSLPVTLGQPASISCKSSQSLVDSDGNTYLNWFQQRPGQSPRRLIYKVSNRDSGVPDRFSGSGSGTDFTLKISRVEAEDVGVYYCMQGTHWPRTFGQGTKVEIKR

SEQ ID NO: 29 - protein, 110 aa, Artificial
  light chain VL amino acid sequence of hFab4.7
MTQSPLSLPVTLGQPASISCRSSQSLVHSDGNMYLNWFQQRPGQSPRRLIYKVSNRDSGVPDRFSGSGSGTDFTLKISRVEAEDVGVYYCMQATQPTWTFGQGTKLEIKR

SEQ ID NO: 30 - protein, 105 aa, Artificial
  light chain VL amino acid sequence of hFab4.8
MTQSPSSLSASVGDRVTITCQASQDISNYLNWYQQKPGKAPKLLIYDASNLETGVPSRFSGSGSGTDFTFTISSLQPDDFATYYCQQTYSWPRTFGQGTKLEIKR

SEQ ID NO: 31 - protein, 105 aa, Artificial
  light chain VL amino acid sequence of hFab4.9
MTQSPSSLSASVGHPVTITCRASQSLISYLNWYHQKPGKAPKLLIYAASILQSGVPSRFSGSGSGTDFTLTISSLQPENFASYYCQHTDSFPRTFGHGTKVEIKR

SEQ ID NO: 32 - protein, 108 aa, Artificial
  light chain VL amino acid sequence of hFab4.10
LTQPPSASGTPGQGVTISCRGSTSNIGNNVVNWYQHVPGSAPKLLIWSNIQRPSGIPDRFSGSKSGTSASLAISGLQSEDEAVYYCAVWDDGLAGWVFGGGTTVTVLS

SEQ ID NO: 33 - protein, 105 aa, Artificial
  light chain VL amino acid sequence of hFab4.12
MTQAPVVSVALEQTVRITCQGDSLAIYYDFWYQHKPGQAPVLVIYGKNNRPSGIPHRFSGSSSNTDSLTITGAQAEDEADYYCNSRDSSGNHWVFGGGTNLTVLG

SEQ ID NO: 34 - protein, 110 aa, Artificial
  light chain VL amino acid sequence of hFab4.18
LTQSPLSLPVTLGQPASISCKSNQSLVHSDGNTYLNWFQQRPGQSPRRLIYKVSNRDSGVPDRFSGSGSGTDFTLKINRVEAEDVGVYYCMQGTQWPRTFGQGTKLDIKR

SEQ ID NO: 35 - protein, 237 aa, Artificial
  IF7 heavy chain amino acid sequence
QPAMAEVQLVQSGGAVVQPGRSLRLSCAASGFTFSTYGMHWVRQAPGKGLEWVAAVSYDGSNKYYADFVKGRFTISRDNPKNTLYLQMNSLRADDTAVYYCARRGGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGPQIR

SEQ ID NO: 36 - protein, 214 aa, Artificial
  amino acid sequence of IF7 light chain
LTQPPPASGTPGQRVTISCSGSSSNIGSNTVNWYQQLPGTAPKLLIYGNDQRPSGVPDRFSASKSGTSASLAISGLQSEDEAHYYCAAWDGSLNGGVIFGGGTKVTVLGQPKAAPSVTLFPPSSEELQANKATLVCLISDFYPGAVTVAWKADSSPVKAGVETTKPSKQSNNKYAASSYLSLTPEQWKSHRSYSCQVTHEGSTVEKTVAPTECS

SEQ ID NO: 37 - protein, 11 aa, Artificial
  consensus sequence of CDR1 of 3C12
  X (2)..(2): A or T
  X (7)..(7): K or S or R
  X (8)..(8): N or S
  X (9)..(9): Y or H or W
  X (10)..(10): F or L
RXSQGIXXXXA

SEQ ID NO: 38 - protein, 7 aa, Artificial
  VL consensus sequence of CDR2 of 3C12
  X (2)..(2): T or A
  X (4)..(4): N or S or T
AXSXLQS

SEQ ID NO: 39 - protein, 9 aa, Artificial
  VL consensus sequence of CDR3 of 3C12
  X (2)..(2): Q or K
  X (3)..(3): L or V or C
  X (4)..(4): G or N or D or S
  X (5)..(5): A or S or R
  X (6)..(6): Y or F or A
  X (8)..(8): Y or L
QXXXXXPXT

SEQ ID NO: 40 - protein, 107 aa, Artificial
  VL consensus sequence of 3C12
  X (2)..(2): I or V
  X (3)..(3): V or Q
  X (4)..(4): M or L
  X (10)..(10): L or F or S
  X (15)..(15): L or I or V
  X (20)..(20): T or S
  X (25)..(25): A or T
  X (30)..(30): K or S or R
  X (31)..(31): N or S
  X (32)..(32): Y or H or W
  X (33)..(33): F or L
  X (39)..(39): R or K
  X (43)..(43): A or V
  X (51)..(51): T or A
  X (53)..(53): N or S or T
  X (66)..(66): G or R
  X (70)..(70): E or D or H
  X (79)..(79): Q or H
  X (81)..(81): E or D
  X (83)..(83): F or I or V
  X (90)..(90): Q or K
  X (91)..(91): L or V or C
  X (92)..(92): G or N or D or S
  X (93)..(93): A or S or R
  X (94)..(94): Y or F or A
  X (96)..(96): L or Y
  X (100)..(100): G or Q
  X (103)..(103): K or R
  X (104)..(104): L or V
EXXXTQSPSXLSASXGDRVXITCRXSQGIXXXXAWYQQXPGKXPKLLIYAXSXLQSGVPSRFSGSXSGTXFTLTISSLXPXDXATYYCQXXXXXPXTFGXGTXXELK

SEQ ID NO: 41 - DNA, 1401 bp, Artificial
  3C12 heavy chain
atgggatggagctgtatcatcctcttcttggtagcaacagctacaggtgtccactccgaggtgcagctgcaggagtcggggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagccgaggacacggctatgtattactgtgcgagacactactactacggtatggacgtctggggccaagggaccacgctcaccgtgtcctccgcctccaccaagggcccttccgtgttccctctggccccttcctccaagtccacctccggcggcaccgccgctctgggctgcctggtgaaggactacttccctgagcctgtgaccgtgtcctggaactctggcgccctgacctctggcgtgcacacctttcctgctgtcctgcagtcctccggcctgtactccctgtcctccgtggtgaccgtgccttcctcctccctgggcacccagacctacatctgcaacgtgaaccacaagccttccaacaccaaggtggacaagaaggtggagcctaagtcctgcgacaagacccacacctgccctccctgccctgcccctgagctgctgggcggaccctccgtgttcctgttccctcctaagcctaaggacaccctgatgatctcccggacccctgaggtgacctgcgtggtggtggacgtgtcccacgaggatcctgaggtgaagttcaattggtacgtggacggcgtggaggtgcacaacgccaagaccaagcctcgggaagagcagtacaactccacctaccgggtggtgtctgtgctgaccgtgctgcaccaggactggctgaacggcaaggaatacaagtgcaaggtgtccaacaaggccctgcctgctcccatcgagaagaccatctccaaggccaagggccagcctcgcgagcctcaggtgtataccctgcctccctcccgggacgagctgaccaagaaccaggtgtccctgacctgtctggtgaagggcttctacccttccgatatcgccgtggagtgggagtccaacggccagcctgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtctcctggcaagtga

SEQ ID NO: 42 - DNA, 702 bp, Artificial
  3C12 light chain
atgggctggtcctgcatcatcctgtttctggtggccaccgccaccggcgtgcactccgagatcgtgatgacccagtctccatccctcctgtctgcttctttaggagacagagtcaccatcacttgtcgggccagtcagggcattaagaattattttgcctggtatcagcaaagaccagggaaagcccctaagctcctgatctatgctacatccaatttgcaaagtggggtcccatcacgattcagcggcagtggatctgggacagaattcactctgacaatcagcagcctgcagcctgaagattttgcaacttactattgtcaacaacttggcgcttacccactcactttcggcggggggaccaagctggaactcaagcggaccgtggccgctccttccgtgttcatcttccctccctccgacgagcagctgaagtccggcaccgcctccgtggtgtgcctgctgaacaacttctaccctcgggaggccaaggtgcagtggaaggtggacaacgccctgcagtccggcaactcccaggaatccgtcaccgagcaggactccaaggactctacctactccctgtcctccaccctgaccctgtccaaggccgactacgagaagcacaaggtgtacgcctgcgaagtgacccaccagggcctgtcctctcccgtgaccaagtccttcaaccggggcgagtgctga

SEQ ID NO: 43 - DNA, 702 bp, Artificial
  3C12.B light chain
atgggctggtcctgcatcatcctgtttctggtggccaccgccaccggcgtgcactccgaggttgtgatgacccagtcccccagcttcctgtccgcctctatcggcgaccgggtgtccatcacctgtcggacctcccagggcatctccaaccacctggcctggtatcagcagaagcccggcaaggcccccaagctgctgatctacgccgcctccagcctgcagtccggcgtgccatccagattctccggctccggcagcggcaccgacttcaccctgaccatcagctccctgcagcccgaggatatcgccacctactactgccagcaggtgaactcctacccctacaccttcggccaggggacacgactggagctcaagcggaccgtggccgctccttccgtgttcatcttccctccctccgacgagcagctgaagtccggcaccgcctccgtggtgtgcctgctgaacaacttctaccctcgggaggccaaggtgcagtggaaggtggacaacgccctgcagtccggcaactcccaggaatccgtcaccgagcaggactccaaggactctacctactccctgtcctccaccctgaccctgtccaaggccgactacgagaagcacaaggtgtacgcctgcgaagtgacccaccagggcctgtcctctcccgtgaccaagtccttcaaccggggcgagtgctga

SEQ ID NO: 44 - DNA, 702 bp, Artificial
  3C12C light chain
atgggctggtcctgcatcatcctgtttctggtggccaccgccaccggcgtgcactccgagattgtgctgactcagtccccctccagcctgtccgcctccgtgggcgacagagtgaccatcacctgtcgggcctcccagggcatccggaactacctggcctggtatcagcagaaacccggcaaggtgcccaagctgctgatctacgccacctccaccctgcagtccggcgtgccctcccggttctctggctccagatccggcaccgagttcaccctgaccatctccagcctgcaccccgaggacttcgccacctactactgccagcaggtggaccggttcccctacaccttcggccaggggaccaaggtggaactcaagcggaccgtggccgctccttccgtgttcatcttccctccctccgacgagcagctgaagtccggcaccgcctccgtggtgtgcctgctgaacaacttctaccctcgggaggccaaggtgcagtggaaggtggacaacgccctgcagtccggcaactcccaggaatccgtcaccgagcaggactccaaggactctacctactccctgtcctccaccctgaccctgtccaaggccgactacgagaagcacaaggtgtacgcctgcgaagtgacccaccagggcctgtcctctcccgtgaccaagtccttcaaccggggcgagtgctga

SEQ ID NO: 45 - DNA, 702 bp, Artificial
  3C12D light chain
atgggctggtcctgcatcatcctgtttctggtggccaccgccaccggcgtgcactccgagatccagatgacccagtccccctccagcctgtccgcctctgtgggcgacagagtgaccatcacctgtcgggcctcccagggcatctccagctggctggcctggtatcagcagaagcccggcaaggcccccaagctgctgatctacgccgccagctccctgcagtccggcgtgccatccagattctccggctccggcagcggcaccgagttcaccctgaccatctccagcctgcagcccgacgacttcgccacctactactgccagaagctgtcctcctacccctacaccttcggcggagggaccaaggtggaactcaagcggaccgtggccgctccttccgtgttcatcttccctccctccgacgagcagctgaagtccggcaccgcctccgtggtgtgcctgctgaacaacttctaccctcgggaggccaaggtgcagtggaaggtggacaacgccctgcagtccggcaactcccaggaatccgtcaccgagcaggactccaaggactctacctactccctgtcctccaccctgaccctgtccaaggccgactacgagaagcacaaggtgtacgcctgcgaagtgacccaccagggcctgtcctctcccgtgaccaagtccttcaaccggggcgagtgctga

SEQ ID NO: 46 - DNA, 702 bp, Artificial
  3C12E light chain
atgggctggtcctgcatcatcctgtttctggtggccaccgccaccggcgtgcactccgagattgtgctgactcagtccccctccagcctgtccgcctccgtgggcgacagagtgaccatcacctgtcgggcctcccagggcatctccaactacctggcctggtatcagcagaaacccggcaaggtgcccaagctgctgatctacgccgcctccaccctgcagtccggcgtgccatccagattctccggctccggcagcggcacccacttcaccctgaccatctccagcctgcagcccgaggacgtggccacctactactgccagaagtgcaactccgccccctacaccttcggccaggggaccaaggtggaactcaagcggaccgtggccgctccttccgtgttcatcttccctccctccgacgagcagctgaagtccggcaccgcctccgtggtgtgcctgctgaacaacttctaccctcgggaggccaaggtgcagtggaaggtggacaacgccctgcagtccggcaactcccaggaatccgtcaccgagcaggactccaaggactctacctactccctgtcctccaccctgaccctgtccaaggccgactacgagaagcacaaggtgtacgcctgcgaagtgacccaccagggcctgtcctctcccgtgaccaagtccttcaaccggggcgagtgctga

SEQ ID NO: 47 - DNA, 663 bp, Artificial
  heavy chain Vh region of 1F7
gaggtccagctggtacagtctggtggagccgtggtccagcctgggaggtccctgagactctcctgtgcagcctctggattcaccttcagtacctatggcatgcactgggtccgccaggctccaggcaaggggctggagtgggtggcagctgtatcatatgatggaagtaataaatactatgcagacttcgtgaaggggcgattcaccatctccagagacaatcccaagaacaccctgtatctgcaaatgaacagcctgagagccgatgacacggccgtatattactgtgcccgcagaggtggtcttgatatctggggccaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgca

SEQ ID NO: 48 - DNA, 648 bp, ARtificial
  1F7 light chain
ctgactcagccacccccagcgtctgggacccccgggcagagggtcaccatctcttgttctggaagcagctccaacatcggaagtaatactgtaaactggtaccagcaactcccaggaacggcccccaaactcctcatttatggtaatgatcagcggccctcaggggtccctgaccgattctctgcctccaagtctggcacctcagcctccctggccatcagtgggctccagtctgaggatgaggctcattattattgtgcagcatgggatggcagtctgaatggtggtgtgatattcggcggagggaccaaggtgaccgtcctgggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataataa

SEQ ID NO: 49 - DNA, 648 bp, ARtificial
  hFab4.1 light chain
gtgacgcagccgccctcagcgtctgggacccccgggcagagggtcaccatctcttgttctgggagcagctccaacatcggaactaatcctgtaaactggtaccagcagctcccaggaacggcccccaaactcctcatctatactactgatcagcggccctcaggggtccctgaccgcttctctggctccaagtctggcacctcagcctccctggccatcagtgggctccagtctgaggatgaggctgattattactgtgcagcatgggatgacagcctgagtggcctttatgtcttcgggactgggaccaaggtcaccgtcctcggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctgcagaatgctcttaataa

SEQ ID NO: 50 - DNA, 651 bp, Artificial
  hFab4.2 light chain
atgacccacactccattgtctctgtccgtcacccctggacagccggcctccatctcctgcaagtctagtcagagtctcttgcatagtgatggaaagacctatttgtattggtacctgcagaggccaggccagtctccacagcccctgatctatgaagtttccaaccggttctctggagtgccagataggttcagtggcagcgggtcagggacagatttcacactgaaaatcagccgggtggaggctgaggatgtcggggtttattactgcatgcaaagtctacaactctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 51 - DNA, 654 bp, Artificial
  hFab4.3 light chain
atgactcagtctccactctccctgcccgtcacccttggacagccggcctccatctcctgcaggtctagtcaaagcctcatacacagtgatggaaacacgtacttggattggtttcagcagaggccaggccaatctccaaggcgcctaatttataaggtttctaaccgggactctggggtcccagacagattcagcggcagtgggtccggcactgatttcacactgagaatcagcagggtggaggctgaggatattggggtgtattactgcatgcaagctacacactggcctcggacgttcggccaggggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 52 - DNA, 654 bp, Artificial
  hFab4.4 light chain
atgactcagtctccactctccctgcccgtcacccttggacagccggcctccatctcctgcaggtctagtcaaagcctcgtagacagtgctggaaacaccttcttgcattggtttcaccagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgtatgcaaggtacacactggccccggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 53 - DNA, 654 bp, Artificial
  hFab4.5 light chain
ctgactcagtctccactctccctgcccgtcacccttggacagccggcctccatctcctgcaagtctagtcaaagcctcgtagacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggcctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataa

SEQ ID NO: 54 - DNA, 693 bp, Artificial
  hFab4.7 light chain
atgactcagtctccactctccctgcccgtcacccttggacagccggcctccatctcctgcaggtctagtcaaagcctcgtacacagtgatggaaacatgtacttgaattggtttcagcagaggccaggccaatctccaaggcgcctaatttataaggtttctaaccgggactctggggtcccagacagattcagcggcagtgggtcaggcacagattttacactgaaaatcagcagagtggaggctgaggatgttggggtttattactgcatgcaagctacacagcccacgtggacgttcggccaagggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaaagcagactacgagaaacacaaagtctacgcctgcgaagtcacccatcagggcctgagctcgcccgtcacaaagagcttcaacaggggagagtgttaataaggcgcgccaattctatttcaaggagacagtcataatga

SEQ ID NO: 55 - DNA, 677 bp, Artificial
  hFab4.8 light chain
atgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcaacctgacgattttgcaacttactactgtcaacagacttacagttggcctcggacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaataaggcgcgccaattctatttcaaggagacagtcataatga

SEQ ID NO: 56 - DNA, 639 bp, Artificial
  hFab4.9 light chain
atgacccagtctccatcctccctgtccgcatctgtaggacacccagtcaccatcacttgccgggcaagtcaaagccttatcagctatttaaattggtatcaccagaaaccagggaaagcccctaagctcctgatctatgcggcatccattttgcaaagtggggtcccatcaaggttcagtggcagtggatctgggacagatttcactctcaccatcagcagtctgcaacctgaaaattttgcaagttactactgtcaacataccgacagtttccctcggacgttcggccacgggaccaaggtggaaatcaaacgaactgtggctgcaccatctgtcttcatcttcccgccatctgatgaccagttgaaatctggaactgcctccgttgtgtgcctgctgaataacttctatcccaaaaaggccaaagtacaatggaaggtggataacgccctcgagtcgggtaactcccaggagagtgtcacagagcaggacgtcaaggacagcacctacagcctcagcagcaccctgacgctgagcaaagccggactacgagaaaccaaagtctacgcctgcgaagtcacccatctgcgaactgagctctcccgtcacaaagagcttcacaggggagagtgttaataa

SEQ ID NO: 57 - DNA, 645 bp, Artificial
  hFab4.10 light chain
ctgactcagcccccctcagcgtctgggacccccgggcagggtgtcaccatctcctgtcgtggaagcacctccaacatcggaaataatgttgttaattggtatcaacatgtcccgggatcggcccccaaactcctcatctggagtaatattcagcggccctcagggattcctgaccgattctctggctccaagtctggcacctcagcctccctggccatcagtggacttcagtctgaagatgaggctgtttattactgtgcagtctgggatgacggcctggctggttgggtgttcggcggagggaccacggtgaccgtcctaagtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttcataataa

SEQ ID NO: 58 - DNA, 639 bp, Artificial
  hFab4.12 light chain
atgactcaggcccctgttgtgtcggtggccttggaacaaacagtcaggatcacatgccaaggagacagcctagcaatctattatgatttctggtaccagcacaagccaggacaggcccctgtacttgtcatctatggtaaaaacaaccggccctcagggatcccccaccgattctctggctccagctcatgaaacacagattccttgaccatcactggggctcaggcggaagatgaggctgactattactgtaactcccgggacagcagtggtaaccattgggtgttcggcggagggaccaacctgaccgtcctaggtcaacccaaggctgccccctcggccattctgttcccgccctcctctgaggagcttcaaactaacacggctacatgggtgtgtctcatatttgacttctacccgggagctgtaacagtggccgggaatgcagatggcaaccccgtcaacgccggagtggataccaccaaaccctactgccagaacaacaactactacgcggccagcacctacctgatcatgacgcctgaccagtggaaatcccacttcagctacagctaactcgtcacgcatgaagggagctccgtggacaagaaaatggcccctgcagaatgctcttaataa

SEQ ID NO: 59 - DNA, 654 bp, Artificial
  hFab4.18 light chain
ctgactcagtctccactctccctgcccgtcacccttggacagccggcctccatctcctgcaagtctaatcaaagcctcgtacacagtgatggaaacacctacttgaattggtttcagcagaggccaggccaatctccaaggcgcctaatctataaggtttctaaccgggactctggggtcccagacagattcagcggcagtgggtcaggcactgatttcacactgaaaatcaacagggtggaggctgaggatgttggggtttattactgcatgcaaggtacacagtggcctcggacttttggccaggggaccaagctggacatcaaacgaactgtggctgcaccatctgtcttcatcttcccgccatctgatgagcagttgaaatctggaactgcctctgttgtgtgcctgctgaataacttctatcccacagaggccaaagtacagtggaaggtggataacgccctccaatcgggtaactcccaggagagtgtcacagagcaggacagcaaggacagcacctacagcctcagcagcaccctgacgctgagcaaagcaaactacaagaaacacaaagtctacgcctgcgaagtcacccatcagggcctgacctcgcccgtcacaaagagcttcaacaagggagagtgttaataa